SEQUENCE LISTING (16 sequences)

SEQ ID NO: 1 - DNA, 3767 bp, Mus musculus
gacagataaataggaagagcctgcaactcactggcacttggaggctcccgaaggaggctgcctgtcgcccaagcacagagccacaaaggatgcagtctaggagggaagaatcacaagccctgtaagatgagtggagccaaacccccagccaagcaccaatacagaaccccgggacaatgaggacacccccctgcccatagcttccagtgcagccaccaaaagtgccaaaatggaccagcaaatggcactcacatgggggctgtgctacatggcactggtggctctctgttggggacacggggtgacagaggcagaagaaacggtccctctgaagactctgcagtgctacaatgactacaccaaccacatcatctgcagctgggcggacacagaggatgcccaggggctaatcaacatgaccctctatcaccagctagagaaaaaacagccagtgtcctgtgagctcagtgaggaactcatgtggtcagagtgcccgtcatcccaccgctgtgtgcccagaagatgtgtcatcccctatacacgattttccatcacaaacgaagactactactccttccggccagatagtgatctgggcatccagctcatggtgccacttgcccagaatgtgcagccaccacttcccaagaacgtcagcatcagctcctctgaggatcgtttcctgctggagtggagtgtgtcccttggggatgcccaggtctcctggctttcatcaaaggacatagagtttgaggtggcttataagcggcttcaggactcctgggaggatgcctacagtctccacactagcaaatttcaggtgaatttcgagccaaagctattcctacccaacagcatctatgctgcccgtgtgcgcactcggctgtccccgggttcaagcttgtctgggagacccagcagatggagcccagaggttcactgggactcccagccaggggacaaggcccagccacagaaccttcaatgcttctttgatgggatccagtccctccactgctcctgggaggtgtggacccagacgactggctctgtttcctttgggctcttctatcgccccagccctgtagctccggaggagaaatgctctccggtggtgaaggagccgccgggggccagtgtctacacccgctaccattgcagtctacctgtgcctgagcccagtgcacacagccagtacacagtctctgttaagcacctggaacaagggaagttcatcatgagctataaccacatccagatggagcctccaaccctcaacctgaccaagaacagagacagctacagcctgcattgggaaactcagaagatggcttactcattcattgagcacacattccaggtccagtacaagaagaaatcggacagctgggaggacagcaagacagagaacctagatcgagcccatagcatggacctctcccagctggagccagacacctcatactgcgccagggtgagggtcaagcccatctctaactacgatgggatctggagcaagtggagcgaagagtacacttggaagactgactgggtgatgcccacgctgtggatagtcctcatcctggtctttctcatcctcaccttgctcctgatccttcgctttggctgtgtctctgtatacaggacgtacaggaagtggaaggaaaagatccccaaccccagcaagagcctcctgttccaggatggaggtaaaggtctctggcctcctggcagcatggcagccttcgccactaagaaccccgctctccaggggccacagagcaggcttcttgctgagcaacagggggagtcatatgcacatttggaagacaacaacgtgtcacctctcactatagaggaccctaatataattcgagttccaccatccgggcctgatacaaccccagctgcctcatccgaatccacagagcaacttcccaatgttcaagtagagggaccaactcctaacagacctaggaagcaattacccagctttgacttcaatgggccctacctggggcctccccaatcccactctctgcctgatctcccagaccagctgggttccccccaggtgggtgggagcctgaagccagcactgccaggctccttggagtacatgtgtctgccccctggaggtcaagcgcaactggttccattgtcccaggtgatggggcagggccaggctatggatgtgcagtgtgggtccagcctggagacctcagggagcccttccgtggagccaaaggagaaccctccagttgagctgagcatggaggaacaggaggcacgggacaacccagtgactctgcccataagctctgggggccctgagggcagtatgatggcctctgattatgtcactcctggagatccggtgctcactctgcccacagggcccctgtctacctctctgggcccctctctagggttgccctcagcccaaagcccccgtctctgtcttaagctgcccagggtcccctctggaagcccagctctagggccaccagggtttgaggactatgtggagctgcctccaagtgtgagccaggctgccaagtcccctccaggccatcctgctcctcctgtggcaagcagccccacagtgatcccaggagagcccagggaggaagtgggcccagcatccccacatcccgaaggcctccttgttcttcagcaggttggggactactgcttcctccctggcctgggacctggctccctctcaccacacagtaagccaccctctccaagtctgtgttctgagactgaggacctagtccaggacttgtctgtcaaaaagtttccctatcagcccatgccccaggcgccagccattcagtttttcaagtccctaaagcatcaggactacctgtccctgcccccttgggacaatagccagtctgggaaggtgtgctgagtctgtctcctcccaatctcaccagcagcctggcaccgcagcctgtggtcctcagcctgagcatcaccacagaagcctctctgagttcacactcctccttgctcccagccctgacatggcaataccccacctgtgcttttctcttccccctctctccctcttcccctccctccatgcacccccatctctctctctgcagttttctgccttctatgggaacttccttcctattcctctgacctgttggagtggggatgaggcctgtgagggtatgggctgtttagggtttacaaacctgtgaggccggacggtggtggcgcattcccttaatcccagcactctggaggcagaggcaggcagatttctgagtttgagtccagcctggtctacaaagtgagttccaggacagccaggactatacagagaacccctgtctcgagaaaccaaaaagaaaaaaaaaagaactgctgaggtcggcttgtccaccacaggcattggtaatacagcacatttctggctctgtgtgaaaggtgggagtctcaggtgagccccttgggaggtcagactgtacaattggtcaggctcttgtcgctacaagaatagacatggtcctctatggttgtctgaaactcctggggagacctggggaaccttgtagagggtacttggtaccctccttatatacagaaatcactaatgtggacaagctggacacagctatggccaggctggagggatgagaagcaatgggaaggctgtgtgtctagaactcatggtctcaactgcagacaagcttagttgtggatcctggacagcacacccagatactaagtctagacagctgagc

SEQ ID NO: 2 - DNA, 781 bp, Homo sapiens
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttcta

SEQ ID NO: 3 - DNA, 1363 bp, Mus musculus
gtataaaggccccctggagctgggccctggcagagcccagagctgcagcccagatcacccagaatccatggctcaactttctgcccagaggcgcatgaagctaatggccctgcagctgctgctgtggcaaagtgcactatggtcaggacgagaggccgttcccctggtcactgtcagcgctctgccaccatccctgcctctgccccgaagcttcctgcttaagtccctggagcaagtgaggaagatccaggccagcggctcggtgctgctggagcagttgtgtgccacctacaagctgtgtcaccccgaggagctggtgttgctgggccactctctggggatcccgaaggcttccctgagtggctgctctagccaggccctgcagcagacacagtgcctaagccagctccacagtgggctctgcctctaccaaggtctcctgcaggctctatcgggtatttcccctgccctggcccccaccttggacttgcttcagctggatgttgccaactttgccaccaccatctggcagcagatggaaaacctaggggtggcccctactgtgcagcccacacagagcgccatgccagccttcacttctgccttccagcgccgggcaggaggtgtcctggccatttcgtacctgcagggcttcctggagacggctcgccttgctctgcaccacttggcctagacctgagcagaaagccctttccagatagtttatttatctctatttaatatttatgcatatttaagcctactatttaaagacaaagacgagaaaatggagctctaagcttctagatcattctctccacttccgagttttgttctcctgcttagagcagagagagaaggctcttgtgtcctcctgtggaggccagggaaggagatgggtaaataccaagtattgattcctgctgctgctccaggcacccagttctgtggcagtacccccaaaaaatcagtgagccctgccgtgctgaggcaccatctcaggggggcccaggcagcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggccctgctggcctggaagttggagtggggggcagaggaggcaggaggaagcctggggggggggttggcatggagggaggccttcccatccaccctcaccctccaccccacctgtcactatagccaagcttgcggataataaagtgtggtgttcc

SEQ ID NO: 4 - DNA, 1703 bp, Homo sapiens
aaaacagcccggagcctgcagcccagccccacccagacccatggctggacctgccacccagagccccatgaagctgatgggtgagtgtcttggcccaggatgggagagccgcctgccctggcatgggagggaggctggtgtgacagaggggctggggatccccgttctgggaatggggattaaaggcacccagtgtccccgagagggcctcaggtggtagggaacagcatgtctcctgagcccgctctgtccccagccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgtgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctccaaaaaaaaaaaaaaaaaaa

SEQ ID NO: 5 - DNA, 4169 bp, Mus musculus
gacagagcgacggggaagagagctagcggggacgaccaggcggcccgcttgggggaagggagtcggcggctcagtgggcctctggggtgtagtatgtgtcagtgcctgtgagtgtgtttgtgtgtgtgtatgtctgtgtgtgtctggcggagagccagggtgatttcccataaaccacatgccccgccagcccgcccgcttaaaaggctgtgccgagggctggccagcgaagctcggccaggggaaagtgaaagtttgcctcggtgctctcggtgtcgctgcggctctctgcatcccaggacagcggcgtggccctcgaccggggcgcgggctcttcagccactagcgagcaagggagcgagcgaaccagggcggccaacacgccgtgccgggacccagctgcccgtatgaccgcgcggggcgccgcggggcgctgcccttcttcgacatggctgggctcccggctgctgctggtctgtctcctcatgagcaggagtattgccaaggaggtgtcagaacactgtagccacatgattgggaatggacacctgaaggtcctgcagcagttgatcgacagtcaaatggagacttcatgccagattgcctttgaatttgtagaccaggaacagctggatgatcctgtttgctacctaaagaaggccttttttctggtacaagacataatagatgagaccatgcgctttaaagacaacacccccaatgctaacgccaccgagaggctccaggaactctccaataacctgaacagctgcttcaccaaggactatgaggagcagaacaaggcctgtgtccgaactttccatgagactcctctccagctgctggagaagatcaagaacttctttaatgaaacaaagaatctccttgaaaaggactggaacatttttaccaagaactgcaacaacagctttgctaagtgctctagccgagatgtggtgaccaagcctgattgcaactgcctgtaccctaaagccacccctagcagtgacccggcctctgcctcccctcaccagccccccgccccctccatggcccctctggctggcttggcttgggatgattctcagaggacagagggcagctccctcttgcccagtgagcttccccttcgcatagaggacccaggcagtgccaagcagcgaccacccaggagtacctgccagaccctcgagtcaacagagcaaccaaaccatggggacagactcactgaggactcacaacctcatccttctgcgggggggcccgtccctggggtggaagacattcttgaatcttcactgggcactaactgggtcctagaagaagcttctggagaggctagtgagggatttttgacccaggaagcaaagttttccccctccacgcctgtagggggcagcatccaggcagagactgacagacccagggccctctcagcatctccattccctaaatcaacagaggaccaaaagccagtggatataacagacaggccgttgacagaggtgaaccctatgagacccattggccagacacagaataatactcctgagaagactgatggtacatccacgctgcgtgaagaccaccaggagccaggctctccccatattgcgacaccgaatccccaacgagtcagcaactcagccacccccgttgctcagttactgcttcccaaaagccactcttggggcattgtgctgccccttggggagcttgagggcaagagaagtaccagggatcgaaggagccccgcagagctggaaggaggatcagcaagtgagggggcagccaggcctgtggcccgttttaattccattcctttgactgacacaggccatgtggagcagcatgagggatcctctgacccccagatccctgagtctgtcttccacctgctggtgccgggcatcatcctagtcttgctgactgttgggggcctcctgttctacaagtggaagtggaggagccatcgaaaccctcagacattggattcttctgtggggcgaccagaggacagctccctgacccaggatgaggacagacaggtggaactgccagtatagaaaggattctatgctgggcacacaggactatctctttatggaaggagacatatgggaacatccaccactaccctctcctaccatcttcctgggaatgtggcctaccactaccagagctcctgcctaccaagactggatgaaagaagcagctttgatggggtctttccatcctcacccttagactctcaaccaaagagaaagggctggaggatgccccccacatactgccactatttattgtgggccctggaggctccctgcattggaggaagggcagctcagcagctcaggaccctttcccttaggggctgcttcctcccctcaaaaccagaacctggcaagggactcactagcctggatggcccatgggagaccaggtcagatgagaaggagcagaagagccctgtgcccagaagacccaactggtgccaaggaatcccagcatggacaggcagggacctgtttcccaagaagagagcctgatattcaaagggtgggacagcatctgcccgacttcccgtaaaggcataaaggcacgcagcccaaaagacgggaagaggaggcctttggctgcttgtgttgacagcttaaaggggtctacaccctcaacttgcttaagtgccctctgctgatagccaggaaggagggagaccagccctgcccctcaggacctgacctggctcatgatgccaagaggaagacagagctctagcctcgtcttctcctgcccacagcccctgccagagttcttttgcccagcagaggcacccctcatgaaggaagccattgcactgtgaatactgaacctgcctgctgaacagcctgtcccatccatccctatgagtgaccatccgtccgaatgttctcccacttccttcagcctctcctcggcttcttgcactgagctggcctcacgtgttgactgagggagcccctgagccccaaccttcccctgcctcagcctttgattgtccagggtgaagctgtgggagaaccgcctgggctaccagtcagagctggtctttgggctgtgttccttgcccaggtttctgcatcttgcactttgacattcccaggagggaagtgactagtggaagggagagaggaaggggaggcagagacaaaggccacaggcagagctatgaatgagaatgggtcttgaaaatatgtgtgcacccctaagcttgaaattgatctctatactctagcccctcagccagcctccttcctgttgtctgaaacctggagctaagcaggttgtcctgtcacaagctctggggactgagctccatgctccaaccccaccctcttctgacctttgttctccagacctgacccaggtaggcaagggtaccctcccagtctcacctaccatactgtgccatctctagccaagcaagccaggtttagagaagggtcaaaaaaaaaaaaaaaaaagggttgtttacttccaacttgttctgatgccctctgtttcccaggccaggcttgtctgtggtgacctgggcatgggtgacagggctctcatttgccccttggtctctttatgctgctgagtccccctttcctgccctccctggctactgggtcaataatctttcaggccatgaatctgggaggagagtggtctgtaagctccatcagccctgtcctgagacagcaggggggaaggacactggagactttcttgtggggcttacttagccttctggttacagactatttccatgctagaaaatacatattttaaaatagaaggaaaaacacagaaacaaaacaaaacaaggcattctctacccctccaccttaaacatatattattaaagacagaagagaaaatccaacccattgcaagaagctctttgtgggtgcctggttacatcggagcaggggagcctcaaatccacctttggagccgcccctgtgtgcattaggaacccttctctcctctgagaaagctcagagggagcactgcctcacaaactgtgagactgcgttttttatacttggaagtggtgaattattttatataaggtcatttaaatatctatttaaaaaataggaagctgcttttatatttaataataaaagaagtgcacaagc

SEQ ID NO: 6 - DNA, 2545 bp, Homo sapiens
gagggctggccagtgaggctcggcccggggaaagtgaaagtttgcctgggtcctctcggcgccagagccgctctccgcatcccaggacagcggtgcggccctcggccggggcgcccactccgcagcagccagcgagcgagcgagcgagcgagggcggccgacgcgcccggccgggacccagctgcccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtacagccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtgtagagggaattctaagacccctcaccatcctggacacactcgtttgtcaatgtccctctgaaaatgtgacgcccagccccggacacagtactccagatgttgtctgaccagctcagagagagtacagtgggactgttaccttccttgatatggacagtattcttctatttgtgcagattaagattgcattagtttttttcttaacaactgcatcatactgttgtcatatgttgagcctgtggttctataaaacccctagttccatttcccataaacttctgtcaagccagaccatctctaccctgtacttggacaacttaacttttttaaccaaagtgcagtttatgttcacctttgttaaagccacctttgtggtttctgcccatcacctgaacctactgaagttgtgtgaaatcctaattctgtcatctccgtagccctcccagttgtgcctcctgcacattgatgagtgcctgctgttgtctttgcccatgttgttgatgtagctgtgaccctattgttcctcacccctgccccccgccaaccccagctggcccacctcttccccctcccacccaagcccacagccagcccatcaggaagccttcctggcttctccacaaccttctgactgtcttttcagtcatgccccctgctcttttgtatttggctaatagtatatcaatttgcactt

SEQ ID NO: 7 - DNA, 629 bp, Mus musculus
aaccccttggaggaccagaacgagacaatggttcttgccagctctaccaccagcatccacaccatgctgctcctgctcctgatgctcttccacctgggactccaagcttcaatcagtggccgggatacccaccgtttaaccagaacgttgaattgcagctctattgtcaaggagattatagggaagctcccagaacctgaactcaaaactgatgatgaaggaccctctctgaggaataagagctttcggagagtaaacctgtccaaattcgtggaaagccaaggagaagtggatcctgaggacagatacgttatcaagtccaatcttcagaaacttaactgttgcctgcctacatctgcgaatgactctgcgctgccaggggtcttcattcgagatctggatgactttcggaagaaactgagattctacatggtccaccttaacgatctggagacagtgctaacctctagaccacctcagcccgcatctggctccgtctctcctaaccgtggaaccgtggaatgttaaaacagcaggcagagcacctaaagtctgaatgttcctcatggcccatggtcaaaaggattttacattcctttatgccatcaaatgtcttatcaatttatcta

SEQ ID NO: 8 - DNA, 924 bp, Homo sapiens
cagagccccacgaaggaccagaacaagacagagtgcctcctgccgatccaaacatgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttgagtccaacgtccagctcgttctctgggccttctcaccacagagcctcgggacatcaaaaacagcagaacttctgaaacctctgggtcatctctcacacattccaggaccagaagcatttcaccttttcctgcggcatcagatgaattgttaattatctaatttctgaaatgtgcagctcccatttggccttgtgcggttgtgttctcatttttatcccattgagactatttatttatgtatgtatgtatttatttatttattgcctggagtgtgaactgtatttattttagcagaggagccatgtcctgctgcttctgcaaaaaactcagagtggggtggggagcatgttcatttgtacctcgagttttaaactggttcctagggatgtgtgagaataaactagactctgaac

SEQ ID NO: 9 - DNA, 2478 bp, Mus musculus
gggcagatcctacggaagttatggcaaagccagagcgcctgggtggccggtgatgcatgcggcccctcttgggatggatggaccaggcgtggcgtgggtgagaggagtcagctgcctgaactgccctgcccagcaccggtttgcggccacccggtggatgaccggggtcctgggagtgattatgggtggtgagagccggctcctgctgcagtcccagtcatcatgactacacccacctcccgcagaccatgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataaacaggacatgtagtaacaacctccaagaatctactggttcatatacttggagaggttgaaacccttccagaagttcctggatgcctcctgctcaaataagccaagcagctgagaaatctacagtgaggtatgagatgatggacacagaaatgcagctgactgctgccgtcagcatatacatataaagatatatcaactatacagatttttgtaatgcaatcatgtcaactgcaatgcttttaaaaccgttccaaatgtttctaacactacaaagtctacaaaaagcaaggctatgaagattcagagtcaccactgttttcttagcaaaatgatggtatggttaaacattcattggtgaaccactgggggagtggaactgtcctgttttagactggagatactggagggctcacggtgatggataatgctcttgaaaacaagagtctatcttaaagcagcagcaaaaagaagcttaaggcacttaaggcatcaacaaatgtagttaaatatgaatgtataacacataacttcagtaaagagcatagcagatatttttaaataaaagtatttttaaagatagaaatgcacttattccaaagatactgaaccttagtattcagtcgcttttgacacttgtgtataataaagcttatataactgaattttcaatttgaaaagtatatttttaaaagaataatatatgctagacttttaattaatgtatatgtttaattttggcattctgtctgtctctctgtctctctctctctctctctctctctctacctatctatctatatatataattttcatatactaccaattgcgtactttggatagtgtctctttttaacctaaatgacctttattaacagctgtcaggttcccttactctcgagagtgttcattgctgcactgtcatttgatcccagttttattgaacacatatcctttaacacactcacgtccagatttagcaggagactaggaccctataactttgttaagagagaaaacactaatttcttgttttatagtagggtcttattcgtatctaaggcaggctaggattgcagacatgagccaatatgcttaattagaaacattctttttatgttaaactcatgtcttttacaagatgcctacatatatcctatgtatatgcctgtttaaatccttttttgtaaggtctgctgtcttccttcagttgtaatggaaagaaacactatgttgtggaggccaaatttctgaaagtgataagggtttgcttgtactgaattctcattctccttgctttttccagccacgtgagcatctagctatctatacgctggatgtatttgaccgatgcctgctccactggcacattgcatgtgtggtagccatgccttcttgcttctccttttccccaacccctataatgctctactcagtggtacagatagctgggattatcacaattttgagagaaacaccaattgtttaaagtttgtttcataatcaccatttgcccagaaaacagttctctcaacttgtttgcaacatgtaataatttaagaaactcaattttgttaatggactttcgataacttccttagatatcccacatctcctacgtgtcagtcctttgtcctgaggaactggtaaaatgggtaagcccttagctagcgaactgaaggcattcgcatgtgtaagataatctctatacctgcaaggctgtctggatggctccctaccaatattgaacaatattctgattttggcg

SEQ ID NO: 10 - DNA, 2116 bp, Homo sapiens
acatccgcggcaacgcctccttggtgtcgtccgcttccaataacccagcttgcgtcctgcacacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaataaaataaaataaaaataaattcacctagtctaaggatgctaaaccttagtactgagttacattgtcatttatatagattataacttgtctaaataagtttgcaatttgggagatatatttttaagataataatatatgtttaccttttaattaatgaaatatctgtatttaattttgacactatatctgtatataaaatattttcatacagcattacaaattgcttactttggaatacatttctcctttgataaaataaatgagctatgtattaacaaaaaaaaaaaaaaaaaaaaaaaaaaaaaaaaaaaaaaa

SEQ ID NO: 11 - DNA, 579 bp, Mus musculus
atgggggtgcccgaacgtcccaccctgctgcttttactctccttgctactgattcctctgggcctcccagtcctctgtgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggtga

SEQ ID NO: 12 - DNA, 1340 bp, Homo sapiens
cccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaaaaaaaaaaaa

SEQ ID NO: 13 - DNA, 2419 bp, Mus musculus
gggtcctgtcagcagcgccacaaagtcttcgagaagagaagatggcctgagcactgtcaagccttgccgcgggactcaaacttcatcaggcccacctccaccccagcctacctgtgtggccatcagcacgggtccgctccccaacgccgggttcccctcgatcactgtaactgcgatgctctggatcccttggatactcgaagaccaggccagcgggctgtggatctagattgctgcgctctgcatccggggacagagtccttggcccacctctctcccacccgactctgccgaaagaagcacagaagctcaagccgcctccatggccccaggaaagattcaggggagaggccccatacagggagccacttcagttagacaccctggccagaatggagctgactgatttgctcctggcggccatgcttcttgcagtggcaagactaactctgtccagccccgtagctcctgcctgtgaccccagactcctaaataaactgctgcgtgactcccacctccttcacagccgactgagtcagtgtcccgacgtcgaccctttgtctatccctgttctgctgcctgctgtggactttagcctgggagaatggaaaacccagacggaacagagcaaggcacaggacattctaggggcagtgtcccttctactggagggagtgatggcagcacgaggacagttggaaccctcctgcctctcatccctcctgggacagctttctgggcaggttcgcctcctcttgggggccctgcagggcctcctaggaacccagcttcctctacagggcaggaccacagctcacaaggaccccaatgccctcttcttgagcttgcaacaactgcttcggggaaaggtgcgcttcctgcttctggtagaaggtcccaccctctgtgtcagacggaccctgccaaccacagctgtcccaagcagtacttctcaactcctcacactaaacaagttcccaaacaggacttctggattgttggagacgaacttcagtgtcacagccagaactgctggccctggacttctgagcaggcttcagggattcagagtcaagattactcctggtcagctaaatcaaacctccaggtccccagtccaaatctctggatacctgaacaggacacacggacctgtgaatggaactcatgggctctttgctggaacctcacttcagaccctggaagcctcagacatctcgcccggagctttcaacaaaggctccctggcattcaacctccagggtggacttcctccttctccaagccttgctcctgatggacacacacccttccctccttcacctgccttgcccaccacccatggatctccaccccagctccaccccctgtttcctgacccttccaccaccatgcctaactctaccgcccctcatccagtcacaatgtaccctcatcccaggaatttgtctcaggaaacatagcgcgggcactggcccagtgagcgtctgcagcttctctcggggacaagcttccccaggaaggctgagaggcagctgcatctgctccagatgttctgctttcacctaaaaggccctggggaagggatacacagcactggagattgtaaaattttaggagctatttttttttaacctatcagcaatattcatcagagcagctagcgatctttggtctattttcggtataaatttgaaaatcactaattctctatatgtgctttcatacattaagtctgcaaatgcctaggcaggtcttgccttttaacccaggtagatgctacaccatatcagaaaacaaaagggaactttcctttgcttcaagtttaagccttcccgcgcctccaacacccccacaatgccctccttcccttcactgtaattctcagtgagactgtatggtcctcgagatatactgctcttgataaagaattaacaggctatcacttagaaagactgtccctacttaagagacaaaactgaacctgtaagagaataactgggagcacccaaaagccaataaaaatcagggaccaatgttcttcactggggcaacagagctcctggaccctgcctcccaagaaagctaacaggaagcctgggagctccacaccccaggtaaggctgtgcagctggctcagtaaagagcagacttggatgtggcagctgagcaaagagcatcagcagctcagcaggggctcagccaggcctgggctcctgcttccctcctgtggaggtcaggcggaagtgcaggaagtggcaagaggcaggctcctcggctcacacagcaggacaagcacagagcgcttgaaggctctttataatttccacaaatgcacctaaaaagcagccctgtgtggccactccaaactttattggaaccctcccaaatgaaggcagacttataggactttccaacc

SEQ ID NO: 14 - DNA, 1688 bp, Homo sapiens
tcttcctacccatctgctccccagagggctgcctgctgtgcacttgggtcctggagcccttctccacccggatagattcttcacccttggcccgcctttgccccaccctactctgcccagaagtgcaagagcctaagccgcctccatggccccaggaaggattcaggggagaggccccaaacagggagccacgccagccagacaccccggccagaatggagctgactgaattgctcctcgtggtcatgcttctcctaactgcaaggctaacgctgtccagcccggctcctcctgcttgtgacctccgagtcctcagtaaactgcttcgtgactcccatgtccttcacagcagactgagccagtgcccagaggttcaccctttgcctacacctgtcctgctgcctgctgtggactttagcttgggagaatggaaaacccagatggaggagaccaaggcacaggacattctgggagcagtgacccttctgctggagggagtgatggcagcacggggacaactgggacccacttgcctctcatccctcctggggcagctttctggacaggtccgtctcctccttggggccctgcagagcctccttggaacccagcttcctccacagggcaggaccacagctcacaaggatcccaatgccatcttcctgagcttccaacacctgctccgaggaaagacttctggattgttggagacaaacttcactgcctcagccagaactactggctctgggcttctgaagtggcagcagggattcagagccaagattcctggtctgctgaaccaaacctccaggtccctggaccaaatccccggatacctgaacaggatacacgaactcttgaatggaactcgtggactctttcctggaccctcacgcaggaccctaggagccccggacatttcctcaggaacatcagacacaggctccctgccacccaacctccagcctggatattctccttccccaacccatcctcctactggacagtatacgctcttccctcttccacccaccttgcccacccctgtggtccagctccaccccctgcttcctgacccttctgctccaacgcccacccctaccagccctcttctaaacacatcctacacccactcccagaatctgtctcaggaagggtaaggttctcagacactgccgacatcagcattgtctcgtgtacagctcccttccctgcagggcgcccctgggagacaactggacaagatttcctactttctcctgaaacccaaagccctggtaaaagggatacacaggactgaaaagggaatcatttttcactgtacattataaaccttcagaagctatttttttaagctatcagcaatactcatcagagcagctagctctttggtctattttctgcagaaatttgcaactcactgattctctacatgctctttttctgtgataactctgcaaaggcctgggctggcctggcagttgaacagagggagagactaaccttgagtcagaaaacagagaaagggtaatttcctttgcttcaaattcaaggccttccaacgcccccatcccctttactatcattctcagtgggactctgatcccatattcttaacagatctttactcttgagaaatgaataagctttctctcagaaatgctgtccctatacactagacaaaactg

SEQ ID NO: 15 - DNA, 1534 bp, Mus musculus
cgctcttcctttgctgaaggccagcgctgaagacttcagagtcatgagaaggatgcttctgcacttgagtgttctgactctcagctgtgtctgggccactgccatggagattcccatgagcacagtggtgaaagagaccttgacacagctgtccgctcaccgagctctgttgacaagcaatgagacgatgaggcttcctgtccctactcataaaaatcaccagctatgcattggagaaatctttcaggggctagacatactgaagaatcaaactgtccgtgggggtactgtggaaatgctattccaaaacctgtcattaataaagaaatacattgaccgccaaaaagagaagtgtggcgaggagagacggaggacgaggcagttcctggattacctgcaagagttccttggtgtgatgagtacagagtgggcaatggaaggctgaggctgagctgctccatggtgacaggacttcacaatttaagttaaattgtcaacagatgcaaaaaccccacaaaactgtgcaaatgcaagggataccatatgctgtttccatttatatttatgtcctgtagtcagttaaacctatctatgtccatatatgcaaagtgtttaacctttttgtatacgcataaaagaaattcctgtagcgcaggctggcctcaaactggtaatgtagccaaggataaccttgaatttctgatcctcctgcctcctcttcctgaaggctgaggttacagacatgcaccattgccactagttcatgaagtgctggagatggaacccaaggctttgtgcatgttaccaactgagttatactccctccccctcatcctcttcgttgcatcagggtctcaagtattccaggctgactttgaactcagtgtgtagccaagggtgaccctgaactcttggtccagatggacgcaggaggatcacatacccaaccttagcatcctttctcctagcccctttagatagatgatacttaatgactctcttgctgagggatgccacaccggggcttcctgctcctatctaacttcaatttaatacccactagtcaatctctcctcaactccctgctactctccccaaactctagtaagcccacttctatttcttggggagagagaaggttgacttttcttatgtcctatgtatgaatcagactgtgccatgactgtgcctctgtgcctggagcagctggattttggaaaagaaaagggacatctccttgcagtgtgaatgagagccagccacatgctgggccttacttctccgtgtaactgaacttaagaagcaaagtaaataccacaaccttactaccccatgccaacagaaagcataaaatggttgggatgttattcaggtatcagggtcactggagaagcctcccccagtttactccaggaaaaacagatgtatgcttttatttaattctgtaagatgttcatattatttatgatggattcagtaagttaatatttattacaacgtatataatattctaataaagcagaagggacaactc

SEQ ID NO: 16 - DNA, 816 bp, Homo sapiens
atgcactttctttgccaaaggcaaacgcagaacgtttcagagccatgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttgagactaaactggtttgttgcagccaaagattttggaggagaaggacattttactgcagtgagaatgagggccaagaaagagtcaggccttaattttcagtataatttaacttcagagggaaagtaaatatttcaggcatactgacactttgccagaaagcataaaattcttaaaatatatttcagatatcagaatcattgaagtattttcctccaggcaaaattgatatacttttttcttatttaacttaacattctgtaaaatgtctgttaacttaatagtatttatgaaatggttaagaatttggtaaattagtatttatttaatgttatgttgtgttctaataaaacaaaaatagacaactgttc

(19) United States
(12) Patent Application Publication
(10) Pub. No.: US 2006/0134784 A1
(43) Pub. Date: Jun. 22, 2006

(54) Methods and compositions for the growth and maintenance of stem cells

(76) Inventors: Ross S. Basch, New York, NY (US); Simon Karpatkin, New York, NY (US); Rashmi Gupta, Albertson, NY (US)

Correspondence Address: KLAUBER & JACKSON, 411 HACKENSACK AVENUE, HACKENSACK, NJ, 07601, US

(21) Appl. No.: 11/290,626

(22) Filed: Nov. 30, 2005

Related U.S. Application Data
(60) Provisional application No. 60/631,828, filed on Nov. 30, 2004.

(51) Int. Cl. C12N 5/08

(52) U.S. Cl. 435/372

(57) ABSTRACT

The present invention relates to pluripotent stem cells and methods of promoting long term growth of such cells. In particular, the methods described are used to enhance the proliferation of hematopoietic stem cells, hematopoietic precursor cells, hemangioblasts and endothelial stem cells. The present invention also relates to methods of using such cells, comprising introducing such stem cells, into the host. The present invention also relates to pharmaceutical compositions comprising the cells and such compositions may include growth factors or cytokines to allow for further cell growth and/or cellular differentiation. The invention further relates to methods of in vivo administration of a protein or gene of interest comprising transfecting the stem cells grown by the methods described, with a construct comprising DNA which encodes a protein of interest and then introducing the stem cell into the host where the protein or gene of interest is expressed.

### CROSS-REFERENCE TO RELATED APPLICATIONS

The present application is a non-provisional application claiming the priority of copending provisional application Ser. No. 60/631,828, filed Nov. 30, 2004, the disclosure of which is incorporated by reference herein in its entirety. Applicants claim the benefits of this application under 35 U.S.C. §119 (e).

### GOVERNMENT RIGHTS CLAUSE

The research leading to the present invention was supported in part by Grant Nos. P30 CA 16097; R01 HL073713; R01 DA043152; and 2T32 H1007151. Accordingly, the Government has certain rights in the invention.

### FIELD OF THE INVENTION

This invention relates generally to pluripotent stem cells, particularly to methods of promoting the growth and/or maintenance of such stem cells. In particular, the invention relates to methods of promoting the growth and/or maintenance of hematopoietic stem cells and methods of use of such cells. The invention also relates to methods of using the stem cells for treatment of diseases, disabilities or conditions whereby transplantation of such stem cells would be beneficial in ameliorating the symptoms associated with such diseases, disabilities or conditions.

### BACKGROUND OF THE INVENTION

Many of the events that occur within the bone marrow can be modeled in long-term bone marrow cultures (LTBMC) (Taichman, R. S., Reilly, M. J., and Emerson, S. G. (2000), Hematol. 4:421-426; Chaddah, M. R., Wu, D. D., and Phillips, R. A. (1996), Exp. Hematol. 24:497-508; Dexter, T. M. (1979), Acta Haematol. 62:299-305; Gartner, S. and Kaplan, H. S. (1980), Proc. Natl. Acad. Sci. U.S.A 77:4756-4759; Mauch, P., Greenberger, J. S., Botnick, L., Hannon, E., and Hellman, S. (1980), Proc. Natl. Acad. Sci. U.S.A 77:2927-2930; Sutherland, H. J., Hogge, D. E., and Eaves, C. J. (1993), Leukemia 7 Suppl 2:S122-5.:S122-S125).

These cultures are capable of producing stem cells as well as mature granulocytes, monocytes, immature erythrocytes and megakaryocytes and under appropriate conditions lymphoid lineage progenitors develop (Whitlock, C. A., Robertson, D., and Witte, O. N. (1984), J. Immunol. Methods. 67:353-369). Although the cultures faithfully replicate the differentiation of many hematopoietic lineages, they are relatively short-lived. The stem cell compartment of these cultures is rapidly depleted and attempts to achieve expansion of hematopoietic stem cells in culture have met with limited success (Miller, C. L. and Eaves, C. J. (1997), 94:13648; McNiece, I. and Briddell, R. (2001), Exp. Hematol. 29:3-11).

These cultures accumulate large numbers of granulocytes and monocytes which are capable of producing significant levels of reactive oxygen species (ROS) including hydrogen peroxide and these ROS may limit the life expectancy of the cultures (Meagher, R. C., Salvado, A. J., and Wright, D. G. (1988); Blood 72:273-281).

Historically ROS have been of interest because of their ability to produce oxidative damage in biological macromolecules including proteins as well as DNA and lipids. These oxidative modifications can both produce necrosis and can provide a strong pro-apoptotic signal leading to cell death in the immediate vicinity of the ROS source (Nardi, M., Tomlinson, S., Greco, M. A., and Karpatkin, S. (2001), Cell 106:551-561; Valencia, A. and Moran, J. (2004)., Free Radic. Biol Med. 36:1112-1125). The toxicity of ROS is modified by cellular enzymes that scavenge ROS and by reactivity with a variety of antioxidants. Recently it has become clear that some ROS, including hydrogen peroxide play a critical role in intracellular signaling. Several signaling pathways have been shown to be sensitive to the level of H₂O₂ in the environment. Many of the signaling effects are mediated through alterations in protein phosphorylatio (O'Loghlen, A., Perez-Morgado, M. I., Salinas, M., and Martin, M. E. (2003), Arch Biochem. Biophys. 417:194-202; van Montfort, R. L., Congreve, M., Tisi, D., Carr, R., and Jhoti, H. (2003), Nature 423:773-777; Lee, K. and Esselman, W. J. (2002), Free Radic. Biol. Med. 33:1121-1132).

H₂O₂ can modulate the function of protein tyrosine phosphatases. Protein tyrosine phosphatases (PTPase) contain a critical cysteine residue in their active site that is a potential target for redox regulation, and which must be in the reduced state for full phosphatase activity (O'Loghlen, A., Perez-Morgado, M. I., Salinas, M., and Martin, M. E. (2003), Arch Biochem. Biophys. 417:194-202; van Montfort, R. L., Congreve, M., Tisi, D., Carr, R., and Jhoti, H. (2003), Nature 423:773-777; Zhanc, Z. Y. (2003), Prog. Nucleic Acid Res Mol. Biol. 73:171-220; Salmeen, A., Andersen, J. N., Myers, M. P., Meng, T. C., Hinks, J. A., Tonks, N. K., and Barford, D. (2003), Nature 423:769-773). For example, H₂O₂ can specifically inhibit the PTPase activity of LAR (leukocyte antigen-related) and protein tyrosine phosphatase-1 (Denu, J. M. and Tanner, K. G. (1998), Biochemistry 37:5633-5642). Inhibition of PTPases through redox modulation may explain the broad spectrum of H₂O₂ on biologic activities. Furthermore, H₂O₂ can modulate the function of transcription factors (Haddad J J, (2002), Biochem Biophys Res Commun., August 30;296(4):847-56; Erratum in: Biochem Biophys Res Commun. (2003), February 7;301(2):625; Li J, Huang B, Shi X, Castranova V, Vallyathan V, Huang C., (2002), Mol Cell Biochem., May-June; 234-235(1-2):161-8; Haddad J J., (2002), Eur Cytokine Netw. April-June; 13(2):250-60; Chung Y W, Jeong D W, Won J Y, Choi E J, Choi Y H, Kim I Y, (2002), Biochem Biophys Res Commun., May 17;293(4):1248-53; Sano M, Fukuda K, Sato T, Kawaguchi H, Suematsu M, Matsuda S, Koyasu S, Matsui H, Yamauchi-Takihara K, Harada M, Saito Y, Ogawa S., (2001), Circ Res., October 12;89(8):661-9; Haddad J J, Land S C., (2001), FEBS Lett., September 14;505(2):269-74; Eyries M, Collins T, Khachigian L M., (2004), Endothelium., March-April; 11(2):133-9).

The earliest precursors of the hematopoietic system as well as the earliest endothelial precursors that will ultimately form the vascular system develop in the blood islands of the embryonic yolk sac (Maximow A A., Arch Mikroskop Anat 73:444, (1909). The fact that the hematopoietic and endothelial lineages develop in close proximity to each other and at precisely the same developmental stage in the blood islands has led to the hypothesis that they develop from a common precursor, a cell known as the hemangioblast. Cells with these properties have been convincingly demonstrated in mouse embryos (Choi K, Kennedy M, Kazarov A, Papadimitriou J C, Keller G., Development 125:725-732, (1998)). A fraction of differentiating embryonic stem (ES) cells begin to express the vascular endothelial growth factor (VEGF) receptor-2 (flk-1) protein. The flk-1⁺ cells include most of the hematopoietic colony-forming activity (Kabrun N, Buhring H J, Choi K, Ullrich A, Risau W, Keller G., Development 124:2039-2048, (1997)) but a subpopulation that expresses both vascular endothelial (VE)-cadherin and flk-1 appears to be even more immature and has the potential to produce both endothelial and erythro-myeloid cells in vitro (Goodrich J A, Cutler G, Tjian R., Cell 84:825-830, (1996); Pelosi E et al., Blood 100:3203-3208, (2002)). Recent evidence indicates that the hematopoietic cell progenitor population found in adult animals (including humans), also contains endothelial stem/progenitor cells and that these may circulate under normal steady-state conditions (Asahara T et al, Science 275:964-967, (1997); Nieda M, Nicol A, Denning-Kendall P, Sweetenham J, Bradley B, Hows J., Br J Haematol 98:775-777, (1997); Shi Q et al., Blood 92:362-367, (1998); Lin Y, Weisdorf D J, Solovey A, Hebbel R P., J Clin Invest 105:71-77, (2000); Gehling U M et al. Blood 95:3106-3112, (2000); Schatteman G C, Hanlon H D, Jiao C, Dodds S G, Christy B A., J Clin Invest 106:571-578, (2000); Pelosi E et al., Blood 100:3203-3208, (2002)).

Circulating endothelial precursors appear to be a diverse collection of cells that vary widely in their maturity and plasticity. At one extreme, some of the cells are committed endothelial cells shed from the lining of other vessels (Gill M et al., Circ Res 88:167-174, (2001)): at the other extreme are cells that have hematopoietic and endothelial potential (Grant M B et al., Nat Med 8:607-612, (2002)) and may in fact, have even greater “plasticity” (Krause D S et al. Cell 105:369-377, (2001)). Between these extremes lie a collection of as yet uncharacterized intermediates. A series of intermediate cells of increasingly limited developmental potential also separates the primitive pluripotential cell from the mature cells of the peripheral blood (Hodgson G S, Bradley T R., Nature 281:381-382, (1979); Magli M C, Iscove N N, Odartchenko N., Nature 295:527-529, (1982); Eaves C J et al., Blood Cells 18:301-307, (1992); Jones R J, Wagner J E, Celano P, Zicha M S, Sharkis S J. Nature 347:188-189, (1990); Lansdorp P M, Sutherland H J, Eaves C J., J Exp Med 172:363-366, (1990); Miller J S, McCullar V, Punzel M, Lemischka I R, Moore K A., Blood 93:96-106, (1999); Jepsen K et al., Cell 102:753-763, (2000)). Recombinant growth factors and clonal culture systems have made it possible to identify and isolate some committed progenitors, but the most immature cells have proved elusive.

Endothelial precursor cells (EPC) have been presumptively isolated from the bone marrow, systemic circulation and identified in angiogenic beds of mice and humans but definitive identification and characterization of these cells in tumor angiogenesis has proved difficult (Asahara T et al., Science 275:964-967, (1997); Asahara T et al., EMBO J. 18:3964-3972, (1999); Rafii S. J Clin Invest 105:17-19, (2000)). The developmental lineage that produces EPCs has not been firmly established and distinguishing between cells derived from mature endothelial cells (EC) or from EPC in a tumor bed using surface markers is difficult because the progeny all express the same markers (CD31, CD34, Tie-2, Flk-1 and VE-cadherin (Yamashita J et al., Nature 408:92-96, (2000), Asahara T et al., Circ Res 83:233-240, (1998); Peichev M et al., Blood 95:952-958, (2000)).

Endothelial cell development also can be replicated in culture (Pelosi E et al., Blood 100:3203-3208, (2002); Quesenberry P et al., J Cell Biochem 45:273-278, (1991)). These cells are found in exceedingly small numbers in normal tissues and have resisted efforts to expand them in culture (Zanjani E D et al., Blood 24:299-306, (1996); Hoffman R et al. Vox Sang 74 Suppl 2:259-264, (1998); McNiece I, Briddell R., Exp Hematol 29:3-11, (2001)). Thus, the existing methods and procedures for the growth and maintenance of stem cells is not satisfactory to provide the numbers of cells necessary to perform studies needed to better understand the early developmental events. Furthermore, in the instance whereby the use of stem cells is contemplated for treatment of various diseases or disabilities, the present methods available for expansion of the stem cell population are not adequate to provide the number of cells needed.

Accordingly, the methods provided herein have been shown to address this need. The citation of references herein shall not be construed as an admission that such is prior art to the present invention.

### SUMMARY OF THE INVENTION

In its broadest aspect, the present invention relates to methods for culturing and promoting the long term growth and/or maintenance of a population of pluripotent stem cells comprising culturing the cells in a growth medium supplemented with catalase, in an amount sufficient to promote the growth and/or maintenance of these cells. More particularly, the invention relates to methods for promoting long term growth and/or maintenance of hematopoietic stem cells and/or progenitor cells, hemangioblasts and vascular endothelial progenitor cells. The invention further relates to methods of using the stem cells or progenitor cells obtained from the culture for treatment of diseases or conditions which are causally related to or following from the lack or insufficiency of cells of a particular lineage. The invention further relates to therapeutic compositions containing these cells for treating subjects suffering from conditions or diseases resulting from the lack of or an insufficiency of cells of a particular lineage.

Accordingly, a first aspect of the invention provides a method for culturing, growing and/or maintaining stem cells or derivatives thereof comprising the steps of:
(a) providing a population comprised of said stem cells or derivatives thereof; and
(b) stimulating the growth of said stem cells or derivatives thereof by incubation of said stem cells in medium supplemented with a growth promoting and/or maintenance promoting amount of catalase; wherein said stimulating promotes the growth of and maintains more stem cells or derivatives thereof in said population as compared to a population which has not been stimulated with a growth promoting and/or maintenance promoting amount of catalase.

In one particular embodiment, the growth promoting and/or maintenance promoting amount of catalase is about 0.1 μg/ml to about 1 mg/ml. In a more particular embodiment, the growth promoting and/or maintenance promoting amount of catalase is about 1 μg/ml to about 800 μg/ml, preferably 5 μg/ml to about 400 μg/ml, and more preferably about 10 μg/ml to about 200 μg/ml. In another particular embodiment, the catalase-treated cultures accumulate cells with the serological properties of hematopoietic progenitor cells (HPC), endothelial cell precursors (EPC), hemangioblasts and hematopoietic stem cells (HSC). In another particular embodiment, the catalase-treated cultures accumulate either pluripotent lineage committed or pluripotent non-lineage-committed stem cells. In another particular embodiment, the stem cells or derivatives thereof are lineage-committed stem cells. In another particular embodiment, the lineage-committed stem cells or derivatives thereof are hematopoietic stem cells (HSC). In another particular embodiment, the hematopoietic stem cells or derivatives thereof are capable of multilineage repopulation in vitro or in vivo. In yet another particular embodiment, the repopulation results in the growth and expansion of cells selected from the group consisting of granulocytes, monocytes, T cells and B cells. In yet another particular embodiment, the methods of the present invention are utilized to grow hematopoietic stem cells from any mammal, preferably a human, although use of the methods for growing stem cells from other mammals is also conceived. In a particular embodiment, the mammal is a human. In yet another particular embodiment, the mammal is a non-human mammal. In yet another particular embodiment, the mammal is selected from the group consisting of mice, rats, rabbits, goats, sheep, swine, dogs, cats, horses and other domestic or non-domestic mammals.

In yet another particular embodiment, the method results in granulopoiesis during the first week of catalase treatment, followed by a decline by the third week. In yet another particular embodiment, the method further results in a large increase in clonal progenitors (CFU-c). In another particular embodiment, the method results in the detectable levels of cells expressing Sca-1 within the first week of culture of mouse bone marrow cells in the presence of catalase. In yet another particular embodiment, the method further results in about 65 to 90% of mouse bone marrow cells expressing Sca-1. In yet another particular embodiment, the method further results in an increase in the number of mouse bone marrow cells having the Sca-1+/LIN− phenotype after two to three weeks in culture with catalase. In yet another particular embodiment, the method further results in quiescence of hematopoietic stem cells and progenitor cells after about four to five weeks in culture in the presence of catalase. In yet another particular embodiment, the method further results in restoration of hematopoiesis upon removal of catalase. In yet another particular embodiment, the method results in the accumulation of long-term bone marrow cells. In yet another particular embodiment, the mouse cells that accumulate in the long term bone marrow cultures (LTBMC) are Sca-1+, Gr-1−, Mac-1−, Ter119−, CD3− and CD4− (lineage negative or LIN− cells). In yet another particular embodiment, about 15% of the cells in the LTBMC grown in catalase are Sca-1+/LIN−/c-Kit+ and which also express intermediate amounts of FcγR but do not express IL7Rα. In yet another particular embodiment, about 25% of the cells in the LTBMC have a phenotype characteristic of a clonogenic common myeloid progenitor cell. In yet another particular embodiment, the clonogenic common myeloid mouse progenitor cell is Sca-1−/LIN−, FcγR bright and predominantly cKit+. In yet another particular embodiment, the method further results in the absence of cells having the characteristics of a common lymphoid progenitor. In yet another particular embodiment, the common lymphoid progenitor mouse cells are IL7Rα+/Sca-1 low and c-Kit low. In yet another particular embodiment, the stem cells or derivatives thereof grown in the presence of catalase are about 200 to 500 times more plentiful than stem cells grown in the absence of catalase. In yet another particular embodiment, in addition to myeloid progenitors, the Sca-1+/LIN− population contains a large number of cells that express CD31, CD34 and have an active Tie-2 promoter, indicating that they are in the endothelial lineage.

A second aspect of the invention provides an isolated pure population of stem cells or derivatives thereof, grown by the method comprising the steps of:
a) providing a population comprised of said stem cells or derivatives thereof; and
b) stimulating the growth of said stem cells or derivatives thereof by incubation of said stem cells in medium supplemented with a growth promoting and/or maintenance promoting amount of catalase; wherein said stimulating promotes the growth of and maintains more stem cells or derivatives thereof in said population as compared to a population which has not been stimulated with a growth promoting and/or maintenance promoting amount of catalase.

In a particular embodiment, the stem cells or derivatives thereof are isolated from a mammal selected from the group consisting of human and non-human primates, rodents, equines, canines, felines, bovines, porcines, ovines, and lagomorphs. In another particular embodiment, the stem cells or derivatives thereof are derived from an autologous or heterologous donor or from umbilical cord blood. Stem cells (embryonic or adult stem cells) and progenitor cells isolated from any other solid organ (liver, muscle, fat tissue, neural tissue, pancreas, spleen, kidney, thyroid, etc.) are also amenable candidates for culturing under the conditions described herein. Examples of the procedures utilized by those skilled in the art for isolation of such cells can be found in U.S. Pat. Nos. 6,200,806; 6,387,367; 6,610,540; 6,242,579 and 6,129,911, all of which are incorporated by reference in their entireties. The isolation of stem cells from cord blood is described in U.S. Pat. Nos. 6,059,968; 6,179,819; 5,916,202; 5,879,318 and 5,690,646, all of which are also incorporated by reference in their entireties. Examples of the methods used for the isolation of hematopoietic stem cells is also described in U.S. Pat. Nos. 6,627,759; 6,127,135; 5,914,108; 5,763,197 and 5,643,741, all of which are incorporated by reference in their entireties. In yet another particular embodiment, the stem cells or derivatives thereof are capable of differentiating into granulocytes, monocytes, T cells and B cells.

A third aspect of the invention provides a method of treating a subject suffering from a disease or disability which is causally related to or following from the lack or insufficiency of cells of a particular lineage, comprising administering to said subject in need of such treatment the stem cells or derivatives thereof grown by the method comprising the steps of:
a) providing a population comprised of said stem cells or derivatives thereof; and
b) stimulating the growth of said stem cells or derivatives thereof by incubation of said stem cells in medium supplemented with a growth promoting and/or maintenance promoting amount of catalase; wherein said stimulating promotes the growth of and maintains more stem cells or derivatives thereof in said population as compared to a population which has not been stimulated with a growth promoting and/or maintenance promoting amount of catalase.

In one particular embodiment, the stem cells grown by the method described herein are administered to subjects in an amount sufficient to replenish the cells lost due to disease or to the treatment associated with the disease. In another particular embodiment, the treatment replenishes the cells of the hematopoietic lineage. In another particular embodiment, the cells are used to treat subjects suffering from a disease selected from the group consisting of leukemia, lymphoma, anemia, multiple myeloma, inherited blood disorders and diseases or treatments resulting in an immunodeficiency, such as but not limited to AIDS. In yet another particular embodiment, the cells are used to replenish hematopoietic precursor cells or to promote replacement of peripheral blood cells destroyed by exposure of a patient to chemotherapeutic drugs or radiation therapy. In another particular embodiment, the stem cells or derivatives thereof are caused to proliferate and differentiate in vitro prior to being administered. In yet another particular embodiment, the stem cells or derivatives thereof are from a heterologous or an autologous donor. In yet another particular embodiment the donor is a fetus, a juvenile or an adult. In yet another particular embodiment, the source of the stem cells is umbilical cord blood. In Another particular embodiment, the stem cells are administered to a subject in conjunction with other therapeutic regimens. In one embodiment, such regimen includes chemotherapy and/or radiation therapy. In another embodiment, such regimen includes treatment with at least one or more growth factors or cytokines such as, but not limited to, GM-CSF, G-CSF, M-CSF, IL-3, IL-7, EPO, TPO, and IL-5. The treatments may be given concurrently or sequentially.

A fourth aspect of the invention provides a method of treating a disease that results from insufficient growth and/or differentiation of hematopoietic stem cells, hematopoietic progenitors, or a combination thereof, the method comprising administering the stem cells or derivatives thereof prepared by the steps of:
a) providing a population comprised of said stem cells or derivatives thereof; and
b) stimulating the growth of said stem cells or derivatives thereof by incubation of said stem cells in medium supplemented with a growth promoting and/or maintenance promoting amount of catalase; wherein said stimulating promotes the growth of and maintains more stem cells or derivatives thereof in said population as compared to a population which has not been stimulated with a growth promoting and/or maintenance promoting amount of catalase.

In one particular embodiment, the cells may be further treated with additional growth and/or differentiation factors that allow for the further growth and/or differentiation of the stem cells into cells of the hematopoietic lineage. In particular, such treatment includes the use of at least one or more additional growth factors and/or cytokines such as, but not limited to, GM-CSF, G-CSF, M-CSF, IL-3, IL-7, EPO, TPO, and IL-5. In another particular embodiment, the hematopoietic cells have the phenotype and/or functional characteristics of granulocytes/neutrophils, monocytes/macrophages, T cells and/or B cells. In another particular embodiment, these cells are administered to subjects who demonstrate a depletion in granulocytes, monocytes, T cells and/or B cells and who suffer from a disease selected from the group consisting of leukemia, lymphoma, anemia, multiple myeloma, inherited blood disorders and diseases or treatments resulting in an immunodeficiency, such as but not limited to AIDS. In another particular embodiment, the subject demonstrates a depletion of granulocytes/neutrophils, monocytes/macrophages, T cells and/or B cells as a result of treatment with chemotherapy or radiation therapy. In yet another embodiment, the administration of the stem cells grown in medium supplemented with catalase, as described herein, to subjects treated with chemotherapy and/or radiation therapy, results in restoration in hematopoietic progenitor cell number. In yet another embodiment, the administration of the stem cells grown in medium supplemented with catalase, as described herein, to subjects treated with chemotherapy and/or radiation therapy, results in restoration of immune cell number and/or function. In another particular embodiment, the immune cells may be selected from the group selected from T cells, B cells, macrophages/monocytes, and natural killer cells. In another particular embodiment, the stem cells or derivatives thereof are caused to proliferate and differentiate in vitro prior to being administered. In yet another particular embodiment, the stem cells or derivatives thereof are from a heterologous or an autologous donor. In yet another particular embodiment the donor is a fetus, a juvenile or an adult. In yet another particular embodiment, the source of the stem cells is umbilical cord blood. In yet another particular embodiment, the stem cells or derivatives thereof are administered locally to the site of tissue damage. In yet another particular embodiment the stem cells or derivatives thereof are administered in an encapsulation device. In yet another particular embodiment, the stem cell derivatives are obtained by genetic transduction of stem cells.

A fifth aspect of the invention provides a method for treating a blood disorder that results in anemia or in an immunodeficiency in a mammal comprising the steps of:
a. providing a pure population of stem cells grown in the presence of a growth promoting and/or maintenance promoting amount of catalase;
b. genetically transforming said stem cells with a gene encoding a growth factor, or substance that provides for further enhanced proliferation and/or differentiation of the stem cells resulting in a transformed population of stem cells that express said growth factor; and
c. administering an effective amount of said transformed population of stem cells to said mammal.

In one embodiment, the cells are transfected with the genes encoding such growth and/or differentiation factors including, but not limited to, GM-CSF, G-CSF, M-CSF, IL-3, IL-7, EPO, TPO, and IL-5. The GenBank accession numbers for these growth factors, which are incorporated by reference in their entireties, can be found in Table 5. The cells grown by the methods of the present invention may also be transfected with other genes that may be used to treat or prevent disease progression. Such diseases include but are not limited to inherited metabolic diseases, inherited immune disorders or inherited red cell disorders or marrow failure. The inherited metabolic disorders may be selected from, but are not limited to, the group consisting of adrenoleukodystrophy, Hurler's Syndrome, Pompes disease, metachromatic leukodystrophy, and osteopetrosis. The inherited immune cell disorders may be selected from, but are not limited to, the group consisting of Severe Combined Immunodeficiency, ADA deficiency, and Wiskott-Aldrich Syndrome. The inherited red cell disorders may be selected from, but are not limited to, the group consisting of pure red cell aplasia, sickle cell disease, beta thalassemia, aplastic anemia and Fanconi anemia. In addition, it may be possible to correct genetic defects that predispose to malignancy using homologous recombination in the cells grown by the methods of the present invention.

A sixth aspect of the invention provides a method of in vivo administration of a protein or gene of interest comprising the step of transfecting the stem cells grown by the methods of the present invention with a vector comprising DNA or RNA which expresses a protein or gene of interest. In a particular embodiment, the present invention relates to pluripotent stem cells or populations of such cells which have been transformed or transfected and thereby contain and can express a gene or protein of interest. Thus, this invention includes pluripotent stem cells genetically engineered to express a gene or protein of interest. In as much as such genetically engineered stem cells can then undergo lineage-committment, the present invention further encompasses lineage-committed cells, which are derived from a genetically engineered pluripotent stem cell, and which express a gene or protein of interest. The lineage-committed cells may be hematopoietic lineage-committed cells and may be multipotent, and may mature and/or differentiate into granulocytes, monocytes, T cells or B cells.

In one particular embodiment, the invention provides a method of producing a genetically engineered pluripotent stem cell comprising the steps of:

(a) transfecting pluripotent stem cells grown in the presence of a growth promoting amount of catalase with a DNA construct comprising at least one of a marker gene or a gene of interest;

(b) selecting for expression of the marker gene or gene of interest in the pluripotent stem cells;

(c) culturing the stem cells selected in (b) in the presence of a growth promoting amount of catalase.

In yet another particular embodiment, the present invention encompasses genetically engineered pluripotent stem cell(s), including human and non-human cells, produced by the method described herein.

A seventh aspect of the present invention provides a method of tissue repair or transplantation in mammals, comprising administering to a mammal a therapeutically effective amount of pluripotent stem cells grown in the presence of a growth promoting amount of catalase.

An eighth aspect of the invention provides a method of preventing and/or treating cellular debilitations, derangements and/or dysfunctions and/or other disease states in mammals, comprising administering to a mammal a therapeutically effective amount of pluripotent stem cells grown in the presence of a growth promoting amount of catalase. In one embodiment, the present invention provides a method of preventing and/or treating cellular debilitations, derangements and/or dysfunctions and/or other disease states in mammals, comprising administering to a mammal a therapeutically effective amount of a hematopoietic lineage-committed cell derived from the pluripotent stem cells of the present invention. In a particular embodiment, the hematopoietic lineage committed cells are selected from the group consisting of granulocytes/neutrophils, monocytes/macrophages, T cells and B cells. In another particular embodiment, the stem cells grown by the methods described herein may be used to treat inherited red blood cell disorders and marrow failure syndromes, inherited immune disorders or inherited metabolic disorders. In one particular embodiment, the stem cells grown by the methods described herein would be transfected with the gene(s) encoding specific factors or enzymes missing in patients having these inherited disorders and the stem cells would be administered to patients suffering from such disorders. The inherited metabolic disorders may be selected from, but are not limited to the group consisting of adrenoleukodystrophy, Hurler's Syndrome, Pompes disease, metachromatic leukodystrophy, and osteopetrosis. The inherited immune cell disorders may be selected from the group consisting of Severe Combined Immunodeficiency, ADA deficiency, and Wiskott-Aldrich Syndrome. The inherited red cell disorders may be selected from the group consisting of pure red cell aplasia, sickle cell disease, beta thalassemia, aplastic anemia and Fanconi anemia.

The therapeutic methods generally referred to herein include the method for the treatment of various pathologies or other cellular dysfunctions and derangements by the administration of pharmaceutical compositions that may comprise proliferation factors or lineage-commitment factors, alone or in combination with the pluripotent stem cells grown by the methods of the present invention, or cells or tissues derived therefrom, or other similarly effective agents, drugs or compounds.

In a further embodiment, the present invention relates to certain therapeutic methods which would be based upon the activity of the pluripotent stem cells of the present invention, including cells or tissues derived therefrom, or upon agents or other drugs determined to act on any such cells or tissues, including proliferation factors and lineage-commitment factors. One exemplary therapeutic method is associated with the prevention or modulation of the manifestations of conditions causally related to or following from the lack or insufficiency of cells of a particular lineage, and comprises administering the pluripotent stem cells of the present invention, including cells or tissues derived therefrom, either individually or in mixture with proliferation factors or lineage-commitment factors in an amount effective to prevent the development or progression of those conditions in the host.

In a further and particular aspect the present invention includes therapeutic methods, including transplantation of the pluripotent stem cells of the present invention, including lineage-uncommitted populations of cells, lineage-committed populations of cells, tissues and organs derived therefrom, in treatment or alleviation of conditions, diseases, disorders, cellular debilitations or deficiencies which would benefit from such therapy. These methods include the replacement or replenishment of cells, tissues or organs. Such replacement or replenishment may be accomplished by transplantation of the pluripotent stem cells of the present invention or by transplantation of lineage-uncommitted populations of cells, lineage-committed populations of cells, tissues or organs derived therefrom.

Thus, the present invention includes a method of transplanting pluripotent stem cells in a host comprising the step of introducing into the host the pluripotent stem cells of the present invention. In a particular embodiment, the pluripotent stem cells are hematopoietic stem cells (HSC). In another particular embodiment, the HSC undergo differentiation to cells of the myeloid or lymphoid lineage. Such differentiation may occur in vitro prior to the transplant of cells to the subject. In another particular embodiment, the HSC are first implanted into the subject, and the subject is then treated with a growth factor and/or cytokine, or combinations thereof, that permit differentiation in vivo.

In a further aspect this invention provides a method of providing a host with purified pluripotent stem cells comprising the step of introducing into the host the pluripotent stem cells of the present invention. In one embodiment, the cells are provided to the subject in a vehicle that allows for delivery of cells to the site where needed.

A ninth aspect of the invention provides pharmaceutical compositions for use in therapeutic methods which comprise or are based upon the pluripotent stem cells grown by the methods of the present invention, including lineage-uncommitted populations of cells, lineage-committed populations of cells, tissues and organs derived therefrom, along with a pharmaceutically acceptable carrier. Also contemplated are pharmaceutical compositions comprising proliferation factors or lineage commitment factors that act on or modulate the pluripotent stem cells of the present invention and/or the cells, tissues and organs derived therefrom, along with a pharmaceutically acceptable carrier. The pharmaceutical compositions of proliferation factors or lineage commitment factors may further comprise the pluripotent stem cells of the present invention, or cells, tissues or organs derived therefrom. The pharmaceutical compositions may comprise the pluripotent stem cells of the present invention, or cells, tissues or organs derived therefrom, in a polymeric carrier or extracellular matrix.

This invention also provides pharmaceutical compositions for the treatment of cellular debilitation, derangement and/or dysfunction in mammals, comprising:

a) a therapeutically effective amount of the pluripotent stem cells of the present invention; and

b) a pharmaceutically acceptable medium or carrier.

This invention also provides pharmaceutical compositions for the treatment of blood disorders resulting in anemia or in an immune deficiency in mammals, comprising:

a) a therapeutically effective amount of the pluripotent stem cells of the present invention; and

b) a pharmaceutically acceptable medium or carrier.

Pharmaceutical compositions of the present invention also include compositions comprising hematopoietic lineage-committed cell(s) derived from the pluripotent stem cells of the present invention, and a pharmaceutically acceptable medium or carrier. Any such pharmaceutical compositions may further comprise a proliferation factor or lineage-commitment factor.

The present invention naturally contemplates several means or methods for preparation or isolation of the pluripotent stem cells of the present invention including as illustrated herein, and the invention is accordingly intended to cover such means or methods within its scope. Accordingly, the pluripotent stem cells may be obtained from a heterologous or autologous donor, or from umbilical cord blood. Stem cells (embryonic or adult stem cells) and progenitor cells isolated from any other solid organ (liver, muscle, fat tissue, skin, neural tissue, pancreas, spleen, kidney, thyroid, etc.) are also amenable candidates for culturing under the conditions described herein. Examples of the procedures utilized by those skilled in the art for isolation of such cells can be found in U.S. Pat. Nos. 6,200,806; 6,387,367; 6,610,540; 6,242,579 and 6,129,911, all of which are incorporated by reference in their entireties. The isolation of stem cells from cord blood is described in U.S. Pat. Nos. 6,059,968; 6,179,819; 5,916,202; 5,879,318 and 5,690,646, all of which are also incorporated by reference in their entireties. Examples of the methods used for the isolation of hematopoietic stem cells is also described in U.S. Pat. Nos. 6,627,759; 6,127,135; 5,914,108; 5,763,197 and 5,643,741, all of which are incorporated by reference in their entireties.

Other objects and advantages will become apparent from a review of the ensuing detailed description and attendant claims taken in conjunction with the following illustrative drawings. All references cited in the present application are incorporated herein in their entirety.

### BRIEF DESCRIPTION OF THE DRAWINGS

FIG. 1a-1e.: Effect of Catalase Treatment on LTBMC:

FIG. 1a. Non-adherent cells recovered in control and catalase (100 ug/ml) treated cultures (mean per culture±S.D.) Low density, non-adherent cells were cultured as described herein. Each point is the mean of 3-5 replicate cultures. The results illustrated are representative of those found in >10 repetitions of this experiment.

FIG. 1b. Wright-Giemsa stained cytospin of the non-adherent cells removed from the culture after 1 week. There is a preponderance of granulocytes at varying stages of maturation (Original magnification 600×; NA 1.40 using a Leitz ortholux microscope and a Nikon D100 digital camera).

FIG. 1c. Photomicrograph of phase contrast images showing differences in control and catalase (100 ug/ml) treated cultures after 24 days of culture. (original magnifications 100×). The * indicates the “cobblestone” areas. The images were obtained using a Nikon D100 digital camera mounted on a Nikon inverted microscope.

FIG. 1d. CFU-c in the non-adherent cells recovered from control and catalase (100 ug/ml) treated cultures,

FIG. 1e. Wright-Giemsa stained cytospin of the non-adherent cells removed from the culture illustrated in FIGS. 1a and 1d after 3 weeks. Almost all of the cells appear to be mature monocytes and/or macrophages (Original magnification 600× as described above).

FIG. 2a-2b: Restoration of Hematopoiesis in Cultures after the Removal of Catalase

FIG. 2a. Recovery of hematopoiesis after the removal of catalase. Photomicrograph of phase contrast images showing differences between cultures maintained in catalase for 5 weeks and cultures from which catalase was removed after 4 weeks. (original magnifications 100× as described in FIG. 1c above).

FIG. 2b. Recovery of clonal progenitors in LTBMC after the removal of catalase after week 4. The results shown are representative of 3 similar experiments and the data shown are the mean and S.D. of 3-5 replicates.

FIG. 3: SCA-1+/LIN− Cells Accumulate in Catalase Treated LTBMC

Non-adherent cells removed from culture each week were stained with antibodies against murine Sca-1 labeled with phycoerythrin (PE) and a lineage marker cocktail (Gr-1, Mac-1 and Ter-119) labeled with FITC. Plot showing percentage of Sca-1 positive and lineage negative cells at weeks 1, 2 and 3 in catalase treated cultures (right panel) versus control cultures (left panel). The histograms shown are representative of those obtained repeatedly over a 2 year period.

FIG. 4a-4c: Phenotype of Lineage Negative Cells from Catalase Treated LTBMC

Non-adherent cells removed from culture at week 2 were stained with an anti lineage cocktail[FITC], and anti-C-kit [PE}, Sca-1[APC] FcγR[PE-Cy7] and IL7Rα[biotin/strepavidin-tricolor]. Representative histograms are shown but similar results have been obtained repeatedly.

FIG. 4a. Histogram showing the staining with the lineage cocktail in the control and catalase treated cultures. The region labeled LIN− was then used as a gate to analyze the distribution of the other markers.

FIG. 4b. LIN− cells from catalase treated cultures were further analyzed for their expression of Sca-1(x-axis in all 2 dimensional histograms) and for the expression of C-kit (panel 1), FcγR (panel 2) and IL7Rα (Panel 3).

FIG. 4c. LIN− cells from control cultures analyzed as in 3b.

FIG. 5a-5b: Endothelial Markers Expressed by Lineage Negative Cells from Catalase Treated LTBMC

FIG. 5a. Two dimensional histograms [cytograms] of CD31 (Y-AXIS) plotted against CD45 (X-AXIS) after 3 weeks of culture with and without catalase. Sca-1+ cells are shown in red and Sca-1− cells are shown in gray.

FIG. 5b. Two dimensional histograms [cytograms] showing the expression of [A] CD31 vs. CD45, [B] CD31 vs. LN (lineage negative) and [C] c-kits vs. LN by non-adherent cells collected after 3 weeks of culture in the presence of catalase.

FIG. 6a-6b: TIE-2/GFP Expression in Catalase Treated LTBMC

FIG. 6a. Phase and fluorescent phot micrographs of LTBMC initiated on irradiated c57b1/6 stroma, with low-density non-adherent Tie-2/GFP bone marrow cells and cultured for 3 weeks in the presence of catalase. (original magnification 200×; digital images overlaid using Photoshop).

FIG. 6b. In each of the figures cells that express the endogenous fluorescence of GFP have been shown as green dots. GFP negative cells are gray.

FIG. 7a-7b: Sorting of SCA-1+/LIN-Cells from Catalase Treated LTBMC

FIG. 7a. Sorting gates used to define populations of cultured cells expressing Sca-1 and lineage markers.

FIG. 7b. Distribution of cells within sorting gates and clonogenic potential of the sorted cells.

FIG. 8: Multilineage Reconstitution of Myelo-Ablated Mice by BMC Cultured with Catalase 6 Weeks after Transplantation

FACS analysis demonstrating multilineage engraftment of donor derived (CD45.1+) cells in the peripheral blood that are stained with Gr-1PE & Mac-1FITC (left) and B220-PE & CD3 FITC (right).

FIG. 9: Reconstruction of Myelo-Ablated Mice after Secondary Transfer of BMC Obtained from Mice That Had Previously Been Grafted with Cultured Cells

Mice that had been transplanted with either catalase-cultured cells or control cells were sacrificed 30 weeks after transplantation and BMC prepared. Cells from each group of mice were pooled and one million viable BMC were transplanted into irradiated C57B1/6 (Ly5.2) recipients as previously described. After 1 month the recipients were bled and the proportion of culture-derived (Ly5.1) cells in the peripheral blood of each mouse was determined. The difference between the proportion of cultured cells recovered from the catalase-treated and control cultures after the injection of 50,000 cells is significant with a P value of 0.00016 (Student's t-test; 2 tailed, unpaired samples). ⋄ 50,000 cells from Control Culture; Δ 20,000 cells from Control Culture; ♦ 50,000 cells from Catalase Culture; ▴ 20,000 cells from Catalase Culture; * Background; — Mean

FIG. 10: A schematic representation of the points during hematopoiesis and differentiation of hematopoietic progenitor cells where catalase may exert its effect.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. PHames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Definitions

The terms used herein have the meanings recognized and known to those of skill in the art, however, for convenience and completeness, particular terms and their meanings are set forth below.

“Stem Cells” are cells, which are not terminally differentiated and are therefore able to produce cells of other types. Stem cells are divided into three types, including totipotent, pluripotent, and multipotent. “Totipotent stem cells” can grow and differentiate into any cell in the body, and thus can grow into an entire organism. These cells are not capable of self-renewal. In mammals, only the zygote and early embryonic cells are totipotent. “Pluripotent stem cells” are true stem cells, with the potential to make any differentiated cell in the body, but cannot contribute to making the extraembryonic membranes (which are derived from the trophoblast). “Multipotent stem cells” are clonal cells that self-renew as well as differentiate to regenerate adult tissues. “Multipotent stem cells” are also referred to as “unipotent” and can only become particular types of cells, such as blood cells or bone cells. The term “stem cells”, as used herein, refers to pluripotent stem cells capable of self-renewal.

“Umbilical cord blood” is a source of adult stem cells, which are obtained from the blood from the placenta and umbilical cord that are left over after birth. The cells are collected by removing the umbilical cord, cleansing it and withdrawing blood from the umbilical vein. This blood is then immediately analyzed for infectious agents and the tissue-type is determined. Cord blood is stored in liquid nitrogen for later use, when it is thawed and injected through a vein of the patient. This kind of treatment, where the stem cells are collected from another donor, is called allogenic treatment. When the cells are collected from the same patient they will be used on, it is called “autologous”. When the cells are collected from a different patient they will be used on, it is called “heterologous”.

“Adult stem cells” can be found in adult beings. Adult stem cells reproduce daily to provide certain specialized cells, for example 200 billion red blood cells are created each day in the body. Until recently it was thought that each of these cells could produce just one particular type of cell. This is called differentiation. However, in the past few years, evidence has been gathered of stem cells that can transform into several different forms. Bone marrow stem cells are known to be able to transform into liver, nerve, muscle and kidney cells. Stem cells isolated from the bone marrow have been found to be pluripotent. Useful sources of adult stem cells are found in organs throughout the body. In the same way that organs can be transplanted from cadavers, researchers have found that these could be used as a source of stem cells as well. Taking stem cells from the brains of corpses they were able to coax them into dividing into valuable neurons.

“Hematopoiesis” refers to the highly orchestrated process of blood cell development and homeostasis. Prenatally, hematopoiesis occurs in the yolk sack, then liver, and eventually the bone marrow. In normal adults it occurs in bone marrow and lymphatic tissues. All blood cells develop from pluripotent stem cells. Pluripotent cells differentiate into stem cells that are committed to three, two or one hematopoietic differentiation pathway. None of these stem cells are morphologically distinguishable, however.

The term “hematopoietic stem cells” as used in the present invention means multipotent stem cells that are capable of differentiating into all blood cells including erythrocytes, leukocytes and platelets. For instance, the “hematopoietic stem cells” as used in the invention are contained not only in bone marrow but also in umbilical cord blood derived cells.

The term “hematopoietic progenitors”, which is used interchangeably with the term “hematopoietic precursors”, refers to those progenitor or precursor cells which are differentiated further than hematopoietic stem cells but are yet to differentiate into progenitors or precursors of respective blood cell lineages (unipotent precursor cells). For example, the “hematopoietic progenitors” as used in the present invention include granulocyte/macrophage associated progenitors (colony-forming unit granuloyte, macrophage, CFU-GM), erythroid associated progenitors (burst-forming unit erythroid, BFU-E), megakaryocyte associated progenitors (colony-forming unit megakaryocyte, CFU-Mk), and myeloid associated stem cells (colony-forming unit mixed, CFU-Mix).

The term “differentiation” of hematopoietic stem cells and/or hematopoietic progenitors as used in the invention means both the change of hematopoietic stem cells into hematopoietic progenitors and the change of hematopoietic progenitors into unipotent hematopoietic progenitors and/or cells having characteristic functions, namely mature cells including erythrocytes, leukocytes and megakaryocytes. Differentiation of hematopoietic stem cells into a variety of blood cell types involves sequential activation or silencing of several sets of genes. Hematopoietic stem cells choose either a lymphoid or myeloid lineage pathway at an early stage of differentiation.

The activity of “promoting the growth and/or maintenance of” hematopoietic stem cells and/or hematopoietic progenitors as used in the invention means the activity by which hematopoietic stem cells and/or hematopoietic progenitors having the above-described functions are expanded to proliferate those hematopoietic stem cells and/or hematopoietic progenitors which have the same functions. Further, the activity of promoting the differentiation of hematopoietic stem cells and/or hematopoietic progenitors as used in the invention means the activity by which hematopoietic stem cells and/or hematopoietic progenitors are differentiated so that they are changed to those hematopoietic progenitors which have the above-described functions, including myeloid associated stem cells, unipotent progenitors and/or mature blood cells (erythrocytes, leukocytes and megakaryocytes).

“Granulopoiesis” as used herein refers to the proliferation and growth of granulocyte progenitor cells within the bone marrow, which then leads to differentiation of these progenitor cells into fully functional cells of the granulocyte series in the peripheral blood. Mature cells in this series include neutrophils, eosinophils, basophils and monocytes. Granulopoiesis is controlled by a number of substances including granulocyte colony stimulating factors.

“Lineage-commitment” refers to the process by which individual cells commit to subsequent and particular stages of differentiation during the developmental sequence leading to the formation of a life form.

The term “lineage-uncommitted” refers to a characteristic of cell(s) whereby the particular cell(s) are not committed to any next subsequent stage of differentiation of the developmental sequence.

The term “lineage-committed” refers to a characteristic of cell(s) whereby the particular cell(s) are committed to a particular next subsequent stage of differentiation of the developmental sequence. Lineage-committed cells, for instance, can include those cells which can give rise to progeny limited to a single lineage within the bone marrow and blood.

“Progenitor cell(s)” are defined as cells that are lineage-committed, i.e., an individual cell can give rise to progeny limited to a single lineage such as the myeloid or lymphoid lineage. They can also be stimulated by particular growth factors to proliferate. If activated to proliferate, progenitor cells have life-spans limited to 50-70 cell doublings before programmed cell senescence and death occurs.

A “clone” or “clonal population” is a population of cells derived from a single cell or common ancestor by mitosis. A “cell line” is a clone of a primary cell that is capable of stable growth in vitro for many generations.

A cell has been “transformed” or “transfected” or “genetically transduced” by exogenous or heterologous DNA when such DNA has been introduced inside the cell. The transforming or transfecting DNA may or may not be integrated (covalently linked) into chromosomal DNA making up the genome of the cell. In prokaryotes, yeast, and mammalian cells for example, the transforming or transfecting DNA may be maintained on an episomal element such as a plasmid. With respect to eukaryotic cells, a stably transformed or transfected cell is one in which the transforming or transfecting DNA has become integrated into a chromosome so that it is inherited by daughter cells through chromosome replication. This stability is demonstrated by the ability of the eukaryotic cell to establish cell lines or clones comprised of a population of daughter cells containing the transforming or transfecting DNA.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in a feature of pathology such as for example, elevated blood pressure, fever or white cell count as may attend its presence and activity.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

A “growth promoting and/or maintenance promoting effective amount” as used herein refers to the amount of catalase necessary to promote growth of and/or the amount of catalase necessary to maintain the stem cell population in vitro. As relates to the present invention, the growth promoting and/or maintenance promoting amount of catalase is about 0.1 μg/ml to about 1 mg/ml. In a more particular embodiment, the growth promoting and/or maintenance promoting amount of catalase is about 1 μg/ml to about 800 μg/ml, preferably 5 μg/ml to about 400 μg/ml, and more preferably about 10 μg/ml to about 200 μg/ml. It is to be understood that the effective amount may differ depending on the particular cell population and species or source from which the cell was obtained. One of skill in the art would be cognizant of how to determine the optimal amount necessary to achieve the desired effect.

“Derivatives thereof” as used herein refers to a stem cell, which has been modified in some way, for example, genetically altered, to contain a foreign gene for another protein, such as but not limited to, a growth or differentiation factor or other proteins that may aid in further growth and/or differentiation of the stem cells or progenitor cells or to treat the diseases or conditions as described in the present invention.

“Multilineage repopulation” refers to the replenishment of cells having the potential to form cells of different lineages, in the case of the present invention, for example, the hematopoietic stem cells may repopulate the bone marrow and precursor and/or progenitor cells in the granulocyte or lymphocyte series may proliferate. Ultimately, fully differentiated cells of the T cell, B cell, monocyte, and red blood cell lineage may repopulate the mammal under treatment with the stem cells of the present invention.

“Quiescence” as used herein refers to a steady state whereby the cells are at rest and are not actively proliferating.

“Clonal progenitors or CFU-c” refers to a colony forming unit culture, in which granulocyte-macrophage progenitor cells are identified by their ability to give rise to monoclonal colonies in the presence of appropriate stimulators in vitro.

“Long term bone marrow cultures” or “LTBMC” refers to cultures of bone marrow cells grown in vitro for a period of time of about 1-3 months. “Long term bone marrow initiating cells” or “LTBMiC” as used herein, refers to bone marrow cells which initiate and maintain the long term bone marrow cultures for a period of time of about 1-3 months. These cells are human or non-human mammalian cells. These cells are considered to be functionally equivalent to stem cells. In the mouse, these cells are Sca-1+, Gr-1−, Mac-1−, Ter119−, CD3− and CD4− (lineage negative or LIN−) cells. In the human, these cells are Gr-1−, Mac-1−, Ter119−, CD3− and CD4− (lineage negative or LIN−) cells.

“Clonogenic common progenitor cell” includes the “common lymphoid progenitor” and “common myeloid progenitor” populations, which reflect the earliest branch points between the lymphoid and myeloid lineages. The commitment of common myeloid progenitors to either the megakaryocyte/erythrocyte or the granulocyte/macrophage lineages are mutually exclusive events. The common lymphoid progenitors lead to formation of B cells, T cells and natural killer cells, while the common myeloid progenitors lead to formation of red cells, platelets, granulocytes and monocytes.

“Phenotypic characteristics of clonogenic common progenitor cells” includes the phenotypic characteristics of the clonogenic common myeloid and clonogenic common lymphoid progenitor cells. The phenotypic characteristics of clonogenic common myeloid progenitor cells of a mouse include CD45+, Sca-1−, Lineage−, cKit+ FcgammaR+ and the phenotypic characteristics of clonogenic common lymphoid progenitor cells of a mouse include CD45+, Sca-1 low, Lineage−, cKit low I17Ralpha+. The phenotypic characteristics of clonogenic common myeloid progenitor cells of a human include CD45+, Lineage−, cKit+ FcgammaR+ and the phenotypic characteristics of clonogenic common lymphoid progenitor cells of a human include CD45+, Lineage−, cKit low I17Ralpha+.

“Encapsulation device” refers to various polymeric or non-polymeric materials that are useful as a carrier for implantation of cells to a subject in need of cell therapy, or for delivery of genetically engineered cells that carry a gene of interest to the site where needed. A successful encapsulation device must fulfill a number of criteria such as mechanical stability, permeability, and biocompatibility. Encapsulation devices utilize polymeric coatings to form semipermeable barriers that encapsulate transplant cells, allowing them to deliver naturally produced agents into the body while rendering them invisible to a patient's immune system. For example, such devices have been used for islet cell encapsulation for in situ insulin delivery in diabetes treatment.

“Hemangioblasts” as used herein refers to a cell, which is a progenitor of both hematopoietic cells and endothelial cells. These cells are characterized in part by the cell surface markers CD45+, CD31+ and TIE-2+.

“Subject” or “patient” refers to a mammal, preferably a human, in need of treatment for a condition, disorder or disease.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65□C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N} C. below the predicted or determined Tₘ with washes of higher stringency, if desired.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

General Description

In its broadest aspect, the present invention relates to methods for culturing and promoting the long term growth and/or maintenance of a population of pluripotent stem cells comprising culturing the cells in a growth medium supplemented with catalase, in an amount sufficient to promote the growth and/or maintenance of these cells. More particularly, the invention relates to methods for promoting long term growth and/or maintenance of hematopoietic stem cells and/or progenitor cells, hemangioblasts and vascular endothelial progenitor cells. The invention further relates to methods of using the stem cells or progenitor cells obtained from the culture for treatment of diseases or conditions which are causally related to or following from the lack or insufficiency of cells of a particular lineage. The invention further relates to pharmaceutical compositions containing these cells for treating subjects suffering from conditions or diseases resulting from the lack of or an insufficiency of cells of a particular lineage.

In one particular aspect of the invention, a method of culturing the stem cells has been developed, which provides for the promotion of long term growth and/or maintenance of stem cells or derivatives thereof. One of the primary aspects of the culture method that allows for such long term growth is the addition of catalase to the culture medium. The inventors have found that addition of catalase at the concentrations disclosed herein to a culture of stem cells provides for about 200 to 500 times more stem cells than in stem cell cultures which lack catalase.

In one aspect, the catalase-treated cultures accumulate cells with the serological properties of hematopoietic progenitor cells (HPC), endothelial cell precursors (EPC), hemangioblasts and hematopoietic stem cells (HSC). In one particular embodiment, the stem cells or derivatives thereof are pluripotent non-lineage-committed stem cells. In another particular embodiment, the stem cells or derivatives thereof are lineage-committed stem cells. In another particular embodiment, the hematopoietic stem cells or derivatives thereof are capable of multilineage repopulation in vitro or in vivo. In yet another particular embodiment, the repopulation results in the growth and expansion of cells selected from the group consisting of granulocytes, monocytes, T cells and B cells. In another particular embodiment, the repopulation may further result in the growth and expansion of erythrocytes and platelets. In another particular embodiment, the repopulation results in the growth and expansion of cells having a phenotype selected from the group consisting of Gr-1+ cells, Mac-1+ cells, CD3+ cells and B-220+ cells.

The culture method further results in granulopoiesis during the first week of catalase treatment, followed by a decline by the third week. The method further results in a large increase in clonal progenitors (CFU-c). The method also results in a detectable level of cells expressing Sca-1 within the first week of culture in the presence of catalase. The method further results in about 65 to 90% of the cells expressing Sca-1, and further results in an increase in the number of cells having the Sca-1+/LIN− phenotype after two to three weeks in culture with catalase. Subsequent to this time period, eg. about four to five weeks in culture in the presence of catalase, one observes that the stem cells and/or progenitor cells become quiescent. Upon removal of catalase at this time period, hematopoiesis resumes, ie. the cells resume growth. Another aspect of the invention is the accumulation of long-term bone marrow cells. These long-term bone marrow cultures (LTBMC) accumulate cells that are Sca-1+, Gr-1−, Mac-1−, Ter119−, CD3− and CD4− (lineage negative or LIN− cells). About 15% of the cells in LTBMC grown in catalase are Sca-1+/LIN−/c-Kit+ and also express intermediate amounts of FcγR, but do not express IL7Rα. About 25% of the cells in LTBMC have a phenotype characteristic of a clonogenic common myeloid progenitor cell. The clonogenic common myeloid progenitor cell is Sca-1−/LIN−, FcγR bright and predominantly cKit+. The method may further result in the absence of cells having the characteristics of a common lymphoid progenitor in vitro, but these cells upon transfer to an animal result in growth of lymphoid cells in vivo. The common lymphoid progenitor cells are IL7Rα+/Sca-1 low and c-Kit low.

A second aspect of the invention provides an isolated pure population of stem cells or derivatives thereof, grown by the methods described herein. In particular, the invention provides for cells having serological properties of hematopoietic progenitor cells (HPC), endothelial cell precursors (EPC), hemangioblasts and hematopoietic stem cells (HSC)

The stem cells or derivatives thereof are isolated from a mammal selected from the group consisting of human and non-human primates, rodents, equines, canines, felines, bovines, porcines, ovines, and lagomorphs. In one aspect, the stem cells or derivatives thereof are derived from an autologous or heterologous donor or from umbilical cord blood. In another aspect, the stem cells or derivatives thereof are capable of differentiating into granulocytes, monocytes, T cells and B cells.

Another aspect of the present invention provides a method of treating a subject suffering from a disease or disability which is causally related to or following from the lack or insufficiency of cells of a particular lineage, comprising administering to said subject in need of such treatment the stem cells or derivatives thereof grown by the methods described herein. In one particular embodiment, the stem cells grown by the method described herein are administered to subjects in an amount sufficient to replenish the cells lost due to disease or to the treatment associated with the disease. In one embodiment, the cells to be replaced due to illness or disease may be blood cells. The treatment with the cells obtained from the LTBMC may be used to replenish the hematopoietic precursor cells in these patients, thus ultimately leading to replacement of circulating blood cells. Alternatively, patients undergoing chemotherapy or radiation therapy for cancer may have their bone marrow cells destroyed by such therapy, thus leading to the patient experiencing susceptibility to various infectious diseases. These patients may be treated with cells derived from the LTBMC of the present invention. In another particular embodiment, the cells may be used to treat patients suffering from Parkinson's disease, Alzheimer's disease, spinal cord injury, traumatic brain injury, stroke, burns, heart disease, diabetes, osteoarthritis, and rheumatoid arthritis. In another particular embodiment, the treatment replenishes the cells of the hematopoietic lineage. In another particular embodiment, the cells are used to treat subjects suffering from a disease selected from the group consisting of leukemia, lymphoma, anemia, multiple myeloma, inherited blood disorders and diseases or treatments resulting in an immunodeficiency, such as but not limited to AIDS. In another particular embodiment, the stem cells or derivatives thereof are caused to proliferate and differentiate in vitro prior to being administered. In yet another particular embodiment, the stem cells or derivatives thereof are from a heterologous or an autologous donor. In yet another particular embodiment the donor is a fetus, a juvenile or an adult. In yet another particular embodiment, the source of the stem cells is umbilical cord blood. In Another particular embodiment, the stem cells are administered to a subject in conjunction with other therapeutic regimens. In one embodiment, such regimen includes chemotherapy and/or radiation therapy. In another embodiment, such regimen includes treatment with at least one or more growth factors or cytokines such as, but not limited to, GM-CSF, G-CSF, M-CSF, IL-3, IL-7, EPO, TPO, and IL-5. The treatments may be given concurrently or sequentially.

Another aspect of the invention provides a method of treating a disease that results from insufficient growth and/or differentiation of hematopoietic stem cells, hematopoietic progenitors, hemangioblasts, vascular endothelial progenitor cells and/or a combination thereof, the method comprising administering the stem cells, progenitor cells or derivatives thereof prepared by the methods described herein.

In one particular embodiment, the cells may be further treated with additional growth and/or differentiation factors that allow for the further growth and/or differentiation of the stem cells into cells of the hematopoietic lineage. In particular, such treatment includes the use of at least one or more additional growth factors and/or cytokines such as, but not limited to, GM-CSF, G-CSF, M-CSF, IL-3, IL-7, EPO, TPO, and IL-5. In another particular embodiment, the hematopoietic cells have the phenotype and/or functional characteristics of granulocytes/neutrophils, monocytes/macrophages, T cells and/or B cells. In another particular embodiment, these cells are administered to subjects who demonstrate a depletion in granulocytes, monocytes, T cells and/or B cells and who suffer from a disease selected from the group consisting of leukemia, lymphoma, anemia, multiple myeloma, inherited blood disorders and diseases or treatments resulting in an immunodeficiency, such as but not limited to AIDS. In another particular embodiment, the subject demonstrates a depletion of granulocytes/neutrophils, monocytes/macrophages, T cells and/or B cells as a result of treatment with chemotherapy or radiation therapy. In yet another embodiment, the administration of the stem cells grown in medium supplemented with catalase, as described herein, to subjects treated with chemotherapy and/or radiation therapy, results in restoration in hematopoietic precursor/progenitor cell number. In yet another embodiment, the administration of the stem cells grown in medium supplemented with catalase, as described herein, to subjects treated with chemotherapy and/or radiation therapy, results in restoration of immune cell number and/or function. In another particular embodiment, the immune cells may be selected from the group selected from T cells, B cells, macrophages/monocytes, and natural killer cells. In another particular embodiment, the stem cells or derivatives thereof are caused to proliferate and differentiate in vitro prior to being administered. In yet another particular embodiment, the stem cells or derivatives thereof are from a heterologous or an autologous donor. In yet another particular embodiment the donor is a fetus, a juvenile or an adult. In yet another particular embodiment, the source of the stem cells is umbilical cord blood. In yet another particular embodiment, the stem cells or derivatives thereof are administered locally to the site of tissue damage. In yet another particular embodiment the stem cells or derivatives thereof are administered in an encapsulation device. In yet another particular embodiment, the stem cell derivatives are obtained by genetic transduction of stem cells.

The pluripotent stem cell of the present invention may be isolated from the group consisting of non-embryonic, postnatal, or adult tissue, bone marrow, and umbilical cord blood. The stem cells may be isolated from non-human mammals or human mammals.

In a further aspect, the methods of isolating a pluripotent stem cell relate to methods whereby a clonal population of such stem cells is isolated, wherein a single pluripotent stem cell is first isolated and then further cultured and expanded in the presence of catalase to generate a clonal population. A single pluripotent stem cell may be isolated by means of limiting dilution or such other methods as are known to the skilled artisan.

Thus, the present invention also relates to a clonal pluripotent stem cell line developed by such method.

The possibilities for diagnostic and therapeutic use that are raised by the existence and isolation of the pluripotent stem cells of the present invention, derive from the fact that the pluripotent stem cells can be isolated from non-embryonic, postnatal or adult animal cells or tissue and are capable of self regeneration on the one hand and of differentiation to cells of myeloid and lymphoid lineages on the other hand. Thus, cells of any of the myeloid and lymphoid lineages can be provided from a single, self-regenerating source of cells obtainable from an animal source even into and through adulthood. As suggested earlier and elaborated further on herein, the present invention contemplates use of the pluripotent stem cells, including cells or tissues derived therefrom, for instance, in pharmaceutical intervention, methods and therapy, cell-based therapies, gene therapy, isolation and assessment of proliferation or lineage-commitment factors, and in varied studies of development and cell differentiation.

Furthermore, hematopoietic stem cells (HSC) are key targets for gene therapy approaches to treat inherited hematologic disorders, cancers, and chronic viral diseases such as acquired immunodeficiency syndrome. Successful application of stem cell-based gene therapies to treat blood system disorders will require efficient delivery of the therapeutic gene to engrafting HSC, long-term reconstitution of hematopoiesis from transduced HSC, and stable expression of the therapeutic gene in the affected blood cell lineages. Most clinical trials using retroviral vectors to transfer therapeutic genes into long-term multilineage-reconstituting HSC have relied on derivatives of the Moloney murine leukemia virus (MMLV). The MMLV long-terminal repeat (LTR) promoter can direct gene expression in blood, spleen, and thymus cells after the transplantation of transduced bone marrow cells to lethally irradiated mice, and it is active in multiple hematopoietic cell lineages of reconstituted mice, including T cells, B cells, myeloid cells, and erythroid cells. However, the prospects of using hematopoietic stem cells or precursor cells for gene therapy are limited by the number of cells one can obtain from the bone marrow or from cord blood. Thus, the methods of the present invention for growth of these cells will allow for enhancing the number of cells for use in such therapy dramatically.

Not only would the dramatic increase in cell number obtained using the methods of the present invention help in gene therapy techniques, it will also help in situations whereby a subject suffers from a cancer or hyperproliferative condition or from an immunodeficiency disease, such as AIDS. In this situation, the subject is often treated with a chemotherapeutic agent or radiation therapy, which depresses the number of circulating blood cells that play a role in protection from infection from various microbial agents. Moreover, this therapy also reduces the number of bone marrow cells that are needed to restore the depleted peripheral blood cells. Thus, reconstitution of this cell population is necessary to aid in prevention of subsequent microbial infections.

Accordingly, the stem cells grown by the methods of the present invention would be extremely useful as adjuncts in the chemotherapeutic or radiotherapeutic treatment of cancer and other related conditions. A major side effect associated with such chemotherapeutic or radiotherapeutic therapy is myelosuppression which then limits the dose of drug used and/or the frequency of treatment. Deaths due to chemo or irradiation associated myelosuppression are generally due to hemorrhage or sepsis. The hemorrhagic deaths are a result of thrombocytopenia, a drastic decrease in the number of platelets. Septic deaths are a result of neutropenia, a severe drop in neutrophils, the cells which play a major role in recovery from bacterial infections. Septic deaths occur even when patients are treated with antibiotics.

However, it is known that with many cancers, the outcome may be better if one could use a more aggressive therapeutic approach by either increasing the dose or frequency of chemo or radiotherapy. Thus, a method for protecting the bone marrow from cytotoxic agents or accelerating the recovery of bone marrow cells following these regimens may allow for such aggressive therapy. One approach to overcoming the hematologic toxicity associated with cancer therapies is autologous bone marrow transplantation to accelerate recovery of hematopoietic cells. Accordingly, one source of HSCs is from bone marrow. Thus, one can obtain stem cells from this source and enhance their proliferation by growth in catalase as described herein. The bone marrow may be obtained from an autologous donor or a heterologous donor.

More recently, several factors have been identified which are known stimulators of hematopoietic cell growth. These colony stimulating factors (CSFs) act on a variety of bone marrow progenitor cells to accelerate their differentiation into mature, active cell populations. Several of these factors have been cloned and are currently used in cancer patients who have undergone a variety of chemotherapeutic regimens. Results show that these agents, in particular, G-CSF (granulocyte colony stimulating factor) and GM-CSF (granulocyte-macrophage colony stimulating factor), could stimulate a sustained rise in neutrophil counts thus reducing the period of neutropenia after administration of a cytotoxic agent. A reduction in the number of days of neutropenia may prove beneficial not only in terms of hospital costs (by reducing the need for inpatient care) but ultimately in alleviating the morbidity and mortality associated with cancer therapy.

An alternative approach to bone marrow transplantation would be the use of mobilized peripheral blood stem cells, which are then grown by the methods of the present invention. For example, one may envision administration of granulocyte colony stimulating factor (G-CSF) or granulocyte macrophage colony stimulating factor (GM-CSF) to subjects. Upon administration of these colony stimulating factors to the subject, the stem cells would leave the bone marrow and enter the circulation. Thus, at about day 4 or 5 after administration of the colony stimulating factors, peripheral blood would be collected from the subject, the red blood cells would be separated from the remaining cells, and the red cells given back to the subject. The remaining cells, which include HSCs, could either be frozen for later use, or cultured in the presence of catalase for further expansion of the hematopoietic stem cell population. These HSCs could be used in place of a bone marrow transplant. Alternatively, they could be used for transfection with a specific gene for gene therapy.

Another source of hematopoietic stem cells for use as described above is from umbilical cord blood. Such cord blood is readily available, for example, from local blood centers. Such blood can be obtained and the HSCs isolated by methods known to one skilled in the art and expanded using the methods described herein.

Furthermore, the ability to regenerate most human tissues damaged or lost due to trauma or disease is substantially diminished in adults. Every year millions of Americans suffer tissue loss or end-stage organ failure. Tissue loss may result from acute injuries as well as surgical interventions, i.e., amputation, tissue debridement, and surgical extirpations with respect to cancer, traumatic tissue injury, congenital malformations, vascular compromise, elective surgeries, etc. Options such as tissue transplantation and surgical intervention are severely limited by a critical donor shortage and possible long term morbidity. Three general strategies for tissue engineering have been adopted for the creation of new tissue: (1). Isolated cells or cell substitutes applied to the area of tissue deficiency or compromise. (2). Cells placed on or within matrices, in either closed or open systems. (3). Tissue-inducing substances, that rely on growth factors (including proliferation factors or lineage-commitment factors) to regulate specific cells to a committed pattern of growth resulting in tissue regeneration, and methods to deliver these substances to their targets.

A wide variety of transplants, congenital malformations, elective surgeries, diseases, and genetic disorders have the potential for treatment with the pluripotent stem cells of the present invention, including cells or tissues derived therefrom, alone or in combination with proliferation factors, lineage-commitment factors, or genes or proteins of interest. Preferred treatment methods include the treatment of tissue loss where the object is to provide cells directly for transplantation whereupon the tissue can be regenerated in vivo, recreate the missing tissue in vitro and then provide the tissue, or providing sufficient numbers of cells suitable for transfection or transformation for ex vivo or in vivo gene therapy.

As described herein, the stem cells of the present invention have the unique capacity to differentiate into cells of the myeloid and lymphoid lineage. The capacity for such differentiation in vitro (in culture) and in vivo, even to correct defects and function in vivo is demonstrated herein in the Examples provided. Thus, the cells of the present invention may be utilized in transplantation, cell replacement therapy, tissue regeneration, gene therapy, and cell therapies wherein cells and tissues of hematopoietic origin are derived in vivo, ex vivo or in vitro.

The isolation of pluripotent stem cells as tissue source for transplantation therapies, that (a) can be isolated and sorted; (b) has unlimited proliferation capabilities while retaining pluripotentcy; (c) can be manipulated to commit to particular cell lineages; (d) is capable of incorporating into the existing tissue; and (e) can subsequently express the respective cell type, may prove beneficial to therapies that maintain or increase the functional capacity and/or longevity of lost, damaged, or diseased tissues.

In a further embodiment, the present invention relates to certain therapeutic methods which would be based upon the activity of the pluripotent stem cells of the present invention, including cells or tissues derived therefrom, or upon agents or other drugs determined to act on any such cells or tissues, including proliferation factors and lineage-commitment factors. One exemplary therapeutic method is associated with the prevention or modulation of the manifestations of conditions causally related to or following from the lack or insufficiency of cells of a particular lineage, and comprises administering the pluripotent stem cells of the present invention, including cells or tissues derived therefrom, either individually or in mixture with proliferation factors or lineage-commitment factors in an amount effective to prevent the development or progression of those conditions in the host.

In a further and particular aspect the present invention includes therapeutic methods, including transplantation of the pluripotent stem cells of the present invention, including lineage-uncommitted populations of cells, lineage-committed populations of cells, tissues derived therefrom, in treatment or alleviation of conditions, diseases, disorders, cellular debilitations or deficiencies which would benefit from such therapy. These methods include the replacement or replenishment of cells or tissues. Such replacement or replenishment may be accomplished by transplantation of the pluripotent stem cells of the present invention or by transplantation of lineage-uncommitted populations of cells, lineage-committed populations of cells, or tissues derived therefrom.

Thus, the present invention includes a method of transplanting pluripotent stem cells in a host comprising the step of introducing into the host the pluripotent stem cells of the present invention, in particular the hematopoietic stem cells grown by the methods of the present invention.

In a further aspect this invention provides a method of providing a host with purified pluripotent stem cells comprising the step of introducing into the host the pluripotent stem cells of the present invention, in particular the hematopoietic stem cells grown by the methods of the present invention.

In a still further aspect, this invention includes a method of in vivo administration of a protein or gene of interest comprising the step of transfecting the pluripotent stem cells of the present invention with a vector comprising DNA or RNA which expresses a protein or gene of interest.

The present invention provides a method of preventing and/or treating cellular debilitations, derangements and/or dysfunctions and/or other disease states in mammals, comprising administering to a mammal a therapeutically effective amount of pluripotent stem cells, in particular the hematopoietic stem cells grown by the methods of the present invention.

The therapeutic method generally referred to herein could include the method for the treatment of various pathologies or other cellular dysfunctions and derangements by the administration of pharmaceutical compositions that may comprise proliferation factors or lineage-commitment factors, alone or in combination with the pluripotent stem cells of the present invention, or cells or tissues derived therefrom, or other similarly effective agents, drugs or compounds identified for instance by a drug screening assay prepared and used in accordance with a further aspect of the present invention.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

Therapeutic Uses of the Stem Cells

The present invention further contemplates therapeutic/pharmaceutical compositions useful in practicing the therapeutic methods of this invention. A subject therapeutic composition includes, in admixture, a pharmaceutically acceptable excipient (carrier) or media and one or more of the pluripotent stem cells of the present invention, including cells or tissues derived therefrom, alone or in combination with proliferation factors or lineage-commitment factors, as described herein as an active ingredient.

The pluripotent stem cells of the present invention, including cells or tissues derived therefrom, alone or in combination with proliferation factors or lineage-commitment factors, may be prepared in pharmaceutical compositions, with a suitable carrier and at a strength effective for administration by various means to a patient experiencing cellular or tissue loss or deficiency.

It is a still further object of the present invention to provide pharmaceutical compositions for use in therapeutic methods which comprise or are based upon the pluripotent stem cells of the present invention, including lineage-uncommitted populations of cells, lineage-committed populations of cells, and tissues derived therefrom, along with a pharmaceutically acceptable carrier or media. Also contemplated are pharmaceutical compositions comprising proliferation factors or lineage commitment factors that act on or modulate the pluripotent stem cells of the present invention and/or the cells and tissues derived therefrom, along with a pharmaceutically acceptable carrier or media. The pharmaceutical compositions of proliferation factors or lineage commitment factors may further comprise the pluripotent stem cells of the present invention, or cells, tissues or organs derived therefrom.

The pharmaceutical compositions of the present invention may comprise the pluripotent stem cells of the present invention, or cells, and tissues derived therefrom, alone or in a polymeric carrier or extracellular matrix.

Suitable polymeric carriers include porous meshes or sponges formed of synthetic or natural polymers, as well as polymer solutions. One form of matrix is a polymeric mesh or sponge; the other is a polymeric hydrogel. Natural polymers that can be used include proteins such as collagen, albumin, and fibrin; and polysaccharides such as alginate and polymers of hyaluronic acid. Synthetic polymers include both biodegradable and non-biodegradable polymers. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof. Non-biodegradable polymers include polyacrylates, polymethacrylates, ethylene vinyl acetate, and polyvinyl alcohols.

Polymers that can form ionic or covalently crosslinked hydrogels which are malleable are used to encapsulate cells. A hydrogel is a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as Pluronics™ or Tetronics™, polyethylene oxide-polypropylene glycol block copolymers which are crosslinked by temperature or pH, respectively. Other materials include proteins such as fibrin, polymers such as polyvinylpyrrolidone, hyaluronic acid and collagen.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Pharmaceutical Compositions

This invention also provides pharmaceutical compositions for the treatment of cellular debilitation, derangement and/or dysfunction in mammals, comprising:

A. a therapeutically effective amount of the pluripotent stem cells of the present invention; and

B. a pharmaceutically acceptable medium or carrier.

Pharmaceutical compositions of the present invention also include compositions comprising hematopoietic stem cells, hematopoietic precursor cells, hemangioblasts and endothelial precursor cells, and a pharmaceutically acceptable medium or carrier. Any such pharmaceutical compositions may further comprise a proliferation factor or lineage-commitment factor.

The present invention naturally contemplates several means or methods for preparation or isolation of the pluripotent stem cells of the present invention including as illustrated herein, and the invention is accordingly intended to cover such means or methods within its scope.

A variety of administrative techniques may be utilized, among them parenteral techniques such as subcutaneous, intravenous and intraperitoneal injections, catheterizations and the like. The therapeutic factor-containing compositions are conventionally administered intravenously, as by injection of a unit dose, for example. Average quantities of the stem cells or cells may vary and in particular should be based upon the recommendations and prescription of a qualified physician or veterinarian.

The preparation of cellular or tissue-based therapeutic compositions as active ingredients is well understood in the art. Such compositions may be formulated in a pharmaceutically acceptable media. The cells may be in solution or embedded in a matrix.

The preparation of therapeutic compositions with factors, including growth, proliferation or lineage-commitment factors, (such as for instance colony stimulating factors or human growth hormone) as active ingredients is well understood in the art. The active therapeutic ingredient is often mixed with excipients or media which are pharmaceutically acceptable and compatible with the active ingredient. In addition, if desired, the composition can contain minor amounts of auxiliary substances such as wetting or emulsifying agents, pH buffering agents which enhance the effectiveness of the active ingredient.

A factor can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, media, or vehicle.

The compositions are administered in a manner compatible with the dosage formulation, and in a therapeutically effective amount. The quantity to be administered depends, for instance, on the subject and debilitation to be treated, capacity of the subject's organ, cellular and immune system to utilize the active ingredient, and the nature of the cell or tissue therapy, etc. Precise amounts of active ingredient required to be administered depend on the judgment of the practitioner and are peculiar to each individual. However, suitable dosages of a factor may range from about 0.1 to 20, preferably about 0.5 to about 10, and more preferably one to several, milligrams of active ingredient per kilogram body weight of individual per day and depend on the route of administration. Suitable regimes for initial administration and follow on administration are also variable, but can include an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The therapeutic compositions, for instance with a proliferation factor or lineage-commitment factor as active ingredient, may further include an effective amount of the factor, and one or more of the following active ingredients: an antibiotic, a steroid. Exemplary formulations are given below:

### Formulations

Intravenous Formulation I

| Ingredient | mg/ml | |
|---|---|---|
| cefotaxime | 250.0 | |
| Factor | 10.0 | |
| dextrose USP | 45.0 | |
| sodium bisulfite USP | 3.2 | |
| edetate disodium USP | 0.1 | |
| water for injection q.s.a.d. | 1.0 | ml |

Intravenous Formulation II

| Ingredient | mg/ml | |
|---|---|---|
| ampicillin | 250.0 | |
| Factor | 10.0 | |
| sodium bisulfite USP | 3.2 | |
| disodium edetate USP | 0.1 | |
| water for injection q.s.a.d. | 1.0 | ml |

Intravenous Formulation III

| Ingredient | mg/ml | |
|---|---|---|
| gentamicin (charged as sulfate) | 40.0 | |
| Factor | 10.0 | |
| sodium bisulfite USP | 3.2 | |
| disodium edetate USP | 0.1 | |
| water for injection q.s.a.d. | 1.0 | ml |

Intravenous Formulation IV

| Ingredient | mg/ml | |
|---|---|---|
| Factor | 10.0 | |
| dextrose USP | 45.0 | |
| sodium bisulfite USP | 3.2 | |
| edetate disodium USP | 0.1 | |
| water for injection q.s.a.d. | 1.0 | ml |

As used herein, “pg” means picogram, “ng” means nanogram, “ug” or “μg” mean microgram, “mg” means milligram, “μl” or “ul” means microliter, “ml” means milliliter, “l” means liter.

Gene Therapy Using the Stem Cells of the Invention

Another feature of this invention is the expression of the DNA sequences of a gene or protein of interest for delivery of particular genes to a subject in need of particular therapy. In one embodiment, the stem cells are transfected with the genes encoding such growth and/or differentiation factors including, but not limited to, GM-CSF, G-CSF, M-CSF, IL-3, IL-7, EPO, TPO, and IL-5. The cells grown by the methods of the present invention may also be transfected with other genes that may be used to treat or prevent disease progression. Such diseases include but are not limited to inherited metabolic diseases, inherited immune disorders or inherited red cell disorders or marrow failure. The inherited metabolic disorders may be selected from, but are not limited to, the group consisting of adrenoleukodystrophy, Hurler's Syndrome, Pompes disease, metachromatic leukodystrophy, and osteopetrosis. The inherited immune cell disorders may be selected from, but are not limited to, the group consisting of Severe Combined Immunodeficiency, adenosine deaminase (ADA) deficiency, and Wiskott-Aldrich Syndrome. The inherited red cell disorders may be selected from, but are not limited to, the group consisting of pure red cell aplasia, sickle cell disease, beta thalassemia, aplastic anemia and Fanconi anemia. In yet another particular embodiment, the subject is suffering from an inherited or non-inherited hemolytic disorder. In yet another particular embodiment, the hemolytic disorder is caused by defective hemoglobin synthesis, by ineffective erythropoiesis, by excessive hemolysis or a combination thereof. In addition, it may be possible to correct genetic defects that predispose to malignancy using homologous recombination in the cells grown by the methods of the present invention. Other diseases that may be treated by the stem cells grown by the methods described herein include mucopolysaccharidoses (MPS) storage disease, Scheie syndrome, Hunter's syndrome, Sanfilippo syndrome, Morquio syndrome, Maroteaux syndrome, Sly syndrome (beta-glucuronidase deficiency and mucolipidosis. Another leukodystrophy disorder that may be treated using the stem cells grown by the method of the present invention includes Krabbe disease (globoid cell leukodystrophy). Lysosomal storage diseases that may be treated using the stem cells grown by the methods described herein include Gaucher disease, Niemann-Pick disease, Sandhoff disease, Tay-Sachs disease, Wolman disease and Lesch-Nyhan syndrome. It may also be possible to treat renal diseases, liver diseases and diseases of the central nervous system such as stroke, spinal cord injury Alzheimer's disease, Parkinson's disease, ALS, multiple sclerosis, and Huntington's disease using the stem cells grown by the methods described herein. Cancers such as leukemia may also be treated using the stem cells grown by the methods described herein. These include Acute Lymphoblastic Leukemia (ALL), Acute Myelogenous Leukemia (AML), Acute Biphenotypic Leukemia, Acute Undifferentiated Leukemia, Chronic Myelogenous Leukemia (CML), Chronic Lymphocytic Leukemia (CLL), Juvenile Chronic Myelogenous Leukemia (JCML), Juvenile Myelomonocytic Leukemia (JMML). Myelodysplastic Syndromes that may be treated using the stem cells grown by the methods described herein include Refractory Anemia (RA), Refractory Anemia with Ringed Siderblasts (RARS), Refractory Anemia with Excess Blasts (RAEB), Refractory Anemia with Excess Blasts in Transformation (RAEB-T), and Chronic Myelomonocytic Leukemia (CMML). Lymphomas that may be treated using the stem cells grown by the methods described herein include Hodgkin's Lymphoma and Non-Hodgkin's Lymphoma and Burkitt's Lymphoma. Another inherited red cell abnormality that may be treated using the stem cells grown by the methods described herein is Blackfan-Diamond Anemia. Also included are Congenital Dyserythropoietic anemia and Paroxysmal Nocturnal Hemoglobinuria. Inherited Platelet Abnormalities that may be treated using the stem cells grown by the methods described herein include Amegakaryocytosis/Congenital Thrombocytopenia, and Glanzmann Thrombobasthenia. Additional Myeloproliferative disorders that may be treated using the stem cells grown by the methods described herein include Acute Myelofibrosis, Agnogenic Myeloid Metaplasia (Myelofibrosis), Polycythemia Vera, and Essential Thrombocythemia. Other inherited immune system disorders that may be treated using the stem cells grown by the methods described herein include Severe Combined Immunodeficiency Disease (SCID) which is X-linked, SCID with absence of T & B Cells, SCID with absence of T Cells, Normal B Cells and Omenn syndrome. Other inherited immune system disorders that may be treated using the stem cells grown by the methods described herein include neutropenias such as Kostmann syndrome, and Myelokathexis. Other inherited immune system disorders that may be treated using the stem cells grown by the methods described herein include Ataxia-Telangiectasia, Bare Lymphocyte Syndrome, Common Variable Immunodeficiency, DiGeorge syndrome, Leukocyte Adhesion Deficiency, Lymphoproliferative Disorders (LPD), Lymphoproliferative Disorders, X-linked (also known as Epstein-Barr Virus Susceptibility), Phagocyte Disorders, Chediak-Higashi syndrome, Chronic Granulomatous Disorder, Chronic Granulomatous Disease, Neutrophil Actin Deficiency, and Reticular Dysgenesis. Cancers of the bone marrow that may be treated using the stem cells grown by the methods described herein include Multiple Myeloma, Plasma Cell Leukemia, and Waldenstrom's Macroglobulinemia.

As is well known in the art, DNA sequences may be expressed by operatively linking them to an expression control sequence in an appropriate expression vector and employing that expression vector to transform an appropriate unicellular host. Such operative linking of a DNA sequence to an expression control sequence, of course, includes, if not already part of the DNA sequence, the provision of an initiation codon, ATG, in the correct reading frame upstream of the DNA sequence. A wide variety of host/expression vector combinations may be employed in expressing the DNA sequences. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), human cells and plant cells in tissue culture.

It will be understood that not all vectors, expression control sequences and hosts will function equally well to express the DNA sequences. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products. Considering these and other factors a person skilled in the art will be able to construct a variety of vector/expression control sequence/host combinations that will express the DNA sequences of this invention on fermentation or in large scale animal culture.

A DNA sequence can be prepared synthetically rather than cloned. The DNA sequence can be designed with the appropriate codons for the amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient construction of genes, which will express analogs or “muteins”. Alternatively, DNA encoding muteins can be made by site-directed mutagenesis of native genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

Diagnostic Uses of the Stem Cells

The present invention also relates to a variety of diagnostic applications, including methods for detecting the presence of proliferation factors or particular lineage-commitment factors, by reference to their ability to elicit proliferation or particular lineage commitment of pluripotent stem cells, including cells or tissues derived therefrom. The diagnostic utility of the pluripotent stem cells of the present invention extends to the use of such cells in assays to screen for proliferation factors or particular lineage-commitment factors, by reference to their ability to elicit proliferation or particular lineage commitment of pluripotent stem cells, including cells or tissues derived therefrom. Such assays may be used, for instance, in characterizing a known factor, identifying a new factor, or in cloning a new or known factor by isolation of and determination of its nucleic acid and/or protein sequence.

As described in detail above, antibody(ies) to the pluripotent stem cells, including cells and tissues derived therefrom, can be produced and isolated by standard methods including the well known hybridoma techniques. For convenience, the antibody(ies) to the pluripotent stem cells will be referred to herein as Ab₁ and antibody(ies) raised in another species as Ab₂.

The presence of pluripotent stem cells can be ascertained by the usual immunological procedures applicable to such determinations. A number of useful procedures are known. Three such procedures which are especially useful utilize either the pluripotent stem cell labeled with a detectable label, antibody Ab₁ labeled with a detectable label, or antibody Ab₂ labeled with a detectable label. The procedures may be summarized by the following equations wherein the asterisk indicates that the particle is labeled, and “stem cell” stands for the pluripotent stem cell:

A. stem cell*+Ab₁=stem cell*Ab₁

B. stem cell+Ab₁*=stem cellAb₁*

C. stem cell+Ab₁+Ab₂*=stem cellAb₁Ab₂*

The procedures and their application are all familiar to those skilled in the art and accordingly may be utilized within the scope of the present invention. The “competitive” procedure, Procedure A, is described in U.S. Pat. Nos. 3,654,090 and 3,850,752. Procedure C, the “sandwich” procedure, is described in U.S. Pat. Nos. RE 31,006 and 4,016,043. Still other procedures are known such as the “double antibody,” or “DASP” procedure.

In each instance, the stem cell forms complexes with one or more antibody(ies) or binding partners and one member of the complex is labeled with a detectable label. The fact that a complex has formed and, if desired, can then be isolated or the amount thereof can be determined by known methods applicable to the detection of labels. Procedures, for instance, for flourescence activated cell sorting are known in the art and provided herein in the Examples. Cells can also be isolated by adherence to a column to which the antibody has been previously bound or otherwise attached to.

It will be seen from the above, that a characteristic property of Ab₂ is that it will react with Ab₁. This is because Ab₁ raised in one mammalian species has been used in another species as an antigen to raise the antibody Ab₂. For example, Ab₂ may be raised in goats using rabbit antibodies as antigens. Ab₂ therefore would be anti-rabbit antibody raised in goats. For purposes of this description and claims, Ab₁ will be referred to as a primary or anti-stem cell antibody, and Ab₂ will be referred to as a secondary or anti-Ab₁ antibody.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The stem cell or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The invention includes an assay system for screening of potential agents, compounds or drugs effective to modulate the proliferation or lineage-committment of the pluripotent stem cells of the present invention, including cells or tissues derived therefrom. These assays may also be utilized in cloning a gene or polypeptide sequence for a factor, by virtue of the factors known or presumed activity or capability with respect to the pluripotent stem cells of the present invention, including cells or tissues derived therefrom.

The assay system could importantly be adapted to identify drugs or other entities that are capable of modulating the pluripotent stem cells of the present invention, either in vitro or in vivo. Such an assay would be useful in the development of agents, factors or drugs that would be specific in modulating the pluripotent stem cells to, for instance, proliferate or to commit to a particular lineage or cell type. For example, such drugs might be used to facilitate cellular or tissue transplantation therapy.

Thus the present invention contemplates to methods for detecting the presence or activity of an agent which is a lineage-commitment factor comprising the steps of:

A. contacting the pluripotent stem cells of the present invention with a sample suspected of containing an agent which is a lineage-commitment factor; and

B. determining the lineage of the so contacted cells by morphology, mRNA expression, antigen expression or other means;

wherein the lineage of the contacted cells indicates the presence or activity of a lineage-commitment factor in said sample.

The present invention also relates to methods of testing the ability of an agent, compound or factor to modulate the lineage-commitment of a lineage uncommitted cell which comprises

A. culturing the pluripotent tem cells of the present invention in a growth medium which maintains the stem cells as lineage uncommited cells;

B. adding the agent, compound or factor under test; and

C. determining the lineage of the so contacted cells by morphology, mRNA expression, antigen expression or other means.

In a further such aspect, the present invention relates to an assay system for screening agents, compounds or factors for the ability to modulate the lineage-commitment of a lineage uncommitted cell, comprising:

A. culturing the pluripotent stem cells of the present invention in a growth medium which maintains the stem cells as lineage uncommited cells;

B. adding the agent, compound or factor under test; and

C. determining the lineage of the so contacted cells by morphology, mRNA expression, antigen expression or other means.

The invention also relates to a method for detecting the presence or activity of an agent which is a proliferation factor comprising the steps of:

A. contacting the pluripotent stem cells of the present invention with a sample suspected of containing an agent which is a proliferation factor; and

B. determining the proliferation and lineage of the so contacted cells by morphology, mRNA expression, antigen expression or other means;

wherein the proliferation of the contacted cells without lineage commitment indicates the presence or activity of a proliferation factor in said sample.

In a further aspect, the invention includes methods of testing the ability of an agent, compound or factor to modulate the proliferation of a lineage uncommitted cell which comprises

A. culturing the pluripotent stem cells of the present invention in a growth medium which maintains the stem cells as lineage uncommited cells;

B. adding the agent, compound or factor under test; and

C. determining the proliferation and lineage of the so contacted cells by mRNA expression, antigen expression or other means.

The invention further relates to an assay system for screening agents, compounds or factors for the ability to modulate the proliferation of a lineage uncommitted cell, comprising:

A. culturing the pluripotent stem cells of the present invention in a growth medium which maintains the stem cells as lineage uncommited cells;

B. adding the agent, compound or factor under test; and

C. determining the proliferation and lineage of the so contacted cells by mRNA expression, antigen expression or other means.

In a further embodiment of this invention, commercial test kits suitable for use by a medical specialist may be prepared to isolate or determine the presence or absence of pluripotent stem cells, or of a proliferation factor or lineage commitment factor. In accordance with the testing techniques discussed above, one class of such kits will contain at least the labeled stem cell or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the isolation of or demonstration of the presence of pluripotent stem cells, comprising:

(a) a predetermined amount of at least one labeled immunochemically reactive component obtained by the direct or indirect attachment of the pluripotent stem cells or a specific binding partner thereto, to a detectable label;

(b) other reagents; and

(c) directions for use of said kit.

More specifically, the test kit may comprise:

(a) a known amount of the pluripotent stem cells as described above (or a binding partner) generally bound to a solid phase to form an immunosorbent, or in the alternative, bound to a suitable tag, or plural such end products, etc. (or their binding partners) one of each;

(b) if necessary, other reagents; and

(c) directions for use of said test kit.

In a further variation, the test kit may be prepared and used for the purposes stated above, which operates according to a predetermined protocol (e.g. “competitive,” “sandwich,” “double antibody,” etc.), and comprises:

(a) a labeled component which has been obtained by coupling the pluripotent stem cells to a detectable label;

(b) one or more additional immunochemical reagents of which at least one reagent is a ligand or an immobilized ligand, which ligand is selected from the group consisting of:
(i) a ligand capable of binding with the labeled component (a);
(ii) a ligand capable of binding with a binding partner of the labeled component (a);
(iii) a ligand capable of binding with at least one of the component(s) to be determined; and
(iv) a ligand capable of binding with at least one of the binding partners of at least one of the component(s) to be determined; and

(c) directions for the performance of a protocol for the detection and/or determination of one or more components of an immunochemical reaction between the pluripotent stem cells and a specific binding partner thereto.

Further Gene Therapy Techniques for Use with the Stem Cells

Non-limiting examples of techniques which can be used to introduce an expression vector encoding a gene of interest into a stem cell of the present invention include:

Adenovirus-Polylysine DNA Complexes: Naked DNA can be introduced into cells by complexing the DNA to a cation, such as polylysine, which is then coupled to the exterior of an adenovirus virion (e.g., through an antibody bridge, wherein the antibody is specific for the adenovirus molecule and the polylysine is covalently coupled to the antibody) (see Curiel, D. T., et al. (1992) Human Gene Therapy 3:147-154). Entry of the DNA into cells exploits the viral entry function, including natural disruption of endosomes to allow release of the DNA intracellularly. A particularly advantageous feature of this approach is the flexibility in the size and design of heterologous DNA that can be transferred to cells.

Receptor-Mediated DNA Uptake: Naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. Receptors to which a DNA-ligand complex have targeted include the transferrin receptor and the asialoglycoprotein receptor. Additionally, a DNA-ligand complex can be linked to adenovirus capsids which naturally disrupt endosomes, thereby promoting release of the DNA material into the cytoplasm and avoiding degradation of the complex by intracellular lysosomes (see for example Curiel et al. (1991) Proc. Natl. Acad. Sci. USA 88:8850; and Cotten, M. et al. (1992) Proc. Natl. Acad. Sci. USA 89:6094-6098; Wagner, E. et al. (1992) Proc. Natl. Acad. Sci. USA 89:6099-6103). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Liposome-Mediated transfection (“lipofection”): Naked DNA can be introduced into cells by mixing the DNA with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel, F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J. Med. Sci. 298:278; and Gould-Fogerite et al. (1989) Gene 84:429-438.

Direct Injection: Naked DNA can be introduced into cells by directly injecting the DNA into the cells. For an in vitro culture of cells, DNA can be introduced by microinjection, although this not practical for large numbers of cells. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Retroviral Mediated Gene Transfer: Defective retroviruses are well characterized for use in gene transfer for gene therapy purposes (for a review see Miller, A. D. (1990) Blood 76:271). A recombinant retrovirus can be constructed having a nucleic acid encoding a gene of interest (e.g., an antibody homologue) inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art.

Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). While any retrovirus may be utilized, the lentivirus approach allows for delivery to a broad variety of cellular targets, both ex vivo (cell lines, primary cells including stem cells, fertilized oocytes, and blastocysts) and in vivo (e.g., brain, lung, liver). The lentivirus vector-mediated delivery allows for the controllable expression of cellular genes both with a high degree of efficacy and without significant leakiness.

Adenoviral Mediated Gene Transfer: The genome of an adenovirus can be manipulated such that it encodes and expresses a gene product of interest (e.g., an antibody homologue) but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to many other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viral Mediated Gene Transfer: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of the introduced DNA can be detected, for example, by Northern blotting, RNase protection or reverse transcriptase-polymerase chain reaction (RT-PCR). Expression of the introduced gene product (e.g., the antibody homologue) in the cell can be detected by an appropriate assay for detecting proteins, for example by immunohistochemistry.

As will be appreciated by those skilled in the art, the choice of expression vector system will depend, at least in part, on the host cell targeted for introduction of the nucleic acid.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

### Example 1

### Isolation and Propagation of Stem Cells

Methods

Primary Stromal Layer:

The femurs and tibiae of a C57B1/6 mouse are removed and the medullary cavities aseptically flushed with long term bone marrow culture medium using a 22G needle and 6 ml syringe into three 25 cm2 tissue culture flasks. The flasks are incubated at 33° C., 5% CO₂ in a humidified incubator. Half of the media in each flask is replaced with an equal amount of fresh media each week. At the end of the third week the nonadherent cells are removed and the culture trypsinized and replated in the same media at a 1:4 dilution. When the adherent layer reaches confluence the flasks are irradiated (450 cGY) to eliminate residual hematopoietic cells. In some experiments the monolayer was irradiated with 2Gy or was treated with Mitomycin C (10 μg/ml for 1 hour).

Low Density, Nonadherent Bone Marrow Cells:

Bone marrow from the femurs and tibiae of either B6.SJL-Ptprc^{a} Pep3^{b}/BoyJ (Ly5.1) or FVB/N-TgN(Tie2/GFP)287Sato(Tie2/GFP) mice is collected in IMDM medium supplemented with 5% serum. A single cell suspension is prepared by passing the suspension through a 27 ga needle. This suspension is underlayed with an equal volume of ficoll hypaque layer (Histopaque-1077, Sigma, density 1.077) and centrifuged at 2000 rpm, for 20 min. at room temperature. Cells that accumulate at the interface are collected. Cells are counted using a Coulter counter Model Zf. Low density cells are plated at a density of 5×10⁶ cells/ml in IMDM with 5% serum and incubated at 37° C. in 5% CO₂ for 3 hours. Nonadherent cells are collected by removing the medium, washing the plate twice with pre-warmed IMDM media and pooling the washes with the original medium.

Long Term Bone Marrow Culture (LTBMC):

10⁵/ml (total 7 mL/flask) low density, nonadherent mouse bone marrow cells are plated on preformed and irradiated stromal layers in LTBMC media (Stem Cell Technologies, Vancouver, BC. M5300). An appropriate amount of catalase is added and the cultures incubated at 33° C. with 5% CO₂ in a humidified incubator. Additional catalase is added on alternate days to maintain the level of catalase activity in the cultures. The cultures are fed once a week with fresh LTBMC medium after demi-depletion. At the time of feeding, nonadherent cells are collected by centrifugation and resuspended in IMDM medium with 5% serum. All of the experiments reported on here were repeated at least three times. In order to determine if the cultures would recover from catalase treatment the following experiment was performed. After 4 weeks of culture (±catalase) the medium (with the nonadherent cells) was removed. The adherent layer was washed with fresh medium and the non-adherent cells pelleted (1500 rpm×10 min.) and resuspended in fresh medium without catalase. These cells, in fresh catalase-free medium, were returned to flasks in which they had grown. Hematopoiesis was assessed as described above for the next 2 weeks. This experiment was also repeated three times.

Colony Forming Units in Culture (CFU-C):

20,000 and 10,000 nonadherent and adherent cells from control culture flasks and catalase treated flasks or 5000 low density mouse bone marrow cells are plated in methyl cellulose medium (M3434, Stemcell Technologies inc., Vancouver, Canada) in six well tissue culture plates. The plates are placed in a sealed plastic bag along with an open Petri dish containing sterile water and incubated at 37° C., 5% CO₂ for 5 to 17 days and the number of colony forming units are determined.

Immunofluorescence:

10⁵ cultured cells or mouse bone marrow cells in a final volume of 100 μl are incubated with various antibodies [CD45-FITC, CD31-PE, C-kit PE (clones YW 62.3, 390 and 2B8) purchased from Caltag; CD45.1-biotin, Sca1-APC, CD16/32-PEcy7, CD34-biotin, IL7Rα-biotin procured from e-biosciences (clones A20, D7, 93, RAM34, A7R34); Sca1-PE and CD8-FITC purchased from BD-biosciences, San Diego, Calif. (clone D7 and 53)] and a cocktail of antibodies against lineage markers labeled with FITC. Lineage specific antibodies GR1-PE and GR1-Fitc, CD4, B220 are purchased from Caltag laboratories, CA (clone RB6-8C5, CT-CD4, RA3-6B2), Cd11b, Ter-119, CD3 are from E-biosciences, San Diego, Calif. (clone M1/70, TER-119, 145-2C11). All staining is performed at limiting dilution of antibodies for 30 minutes at room temperature. Cells stained with biotinylated antibodies are washed once with PBS and labeled with either avidin-PE-Cy7, avidin-PE-Cy5 or streptavidin-APC. The cells are washed again with PBS and fixed by adding 0.2 ml of 0.2% paraformaldehyde, pH 7.5. In most experiments the cells are analyzed using a FACS Vantage flow cytometer (Becton Dickinson Immunocytometry Systems) but the 5-color analysis is performed using a MoFlo Cytometer and Sorter (Cytomation, Inc. Fort Collins, CO 80525-5576).

Mouse low density bone marrow cells or culture treated cells are sorted in a MoFlo Sorter after staining with Sca1-PE or Lineage-FITC cocktail containing Gr1, CD11b, B220, CD4, CD3, CD8 and TER119 as described above. The stained cells are not fixed and are suspended in PBS containing 5% serum. The sorted cells are collected in PBS+serum.

Repopulation of Lethally Irradiated Mice with Culture Treated Cells:

C57B1/6 mice (Ly5.2) (5-8 mice per group) are irradiated with 1100 cGy (550 cGY×2, 3 hours apart) and then injected intravenously with SJL/pep3b (Ly5.1) BMC from either fresh marrow or adherent and nonadherent cells from control and catalase treated cultures. Mice in all the test groups also received 30,000 LD-syngeneic BMC. After 3, 6 and 10 weeks, the mice are bled and the PBL analyzed for the presence of donor derived lymphocytes (CD3 and B220) and myeloid cells (Gr-1 and Mac-1).

Results

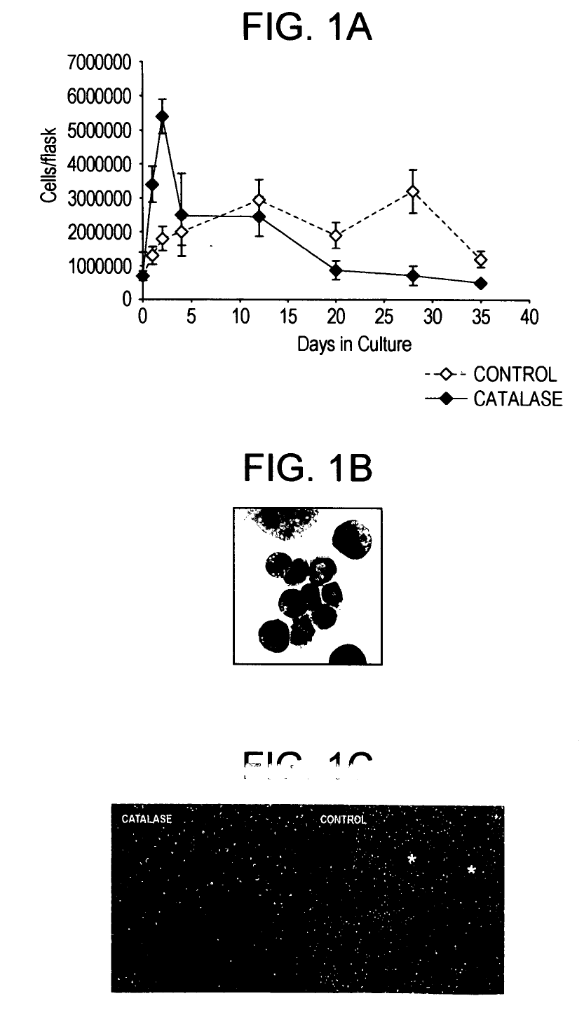

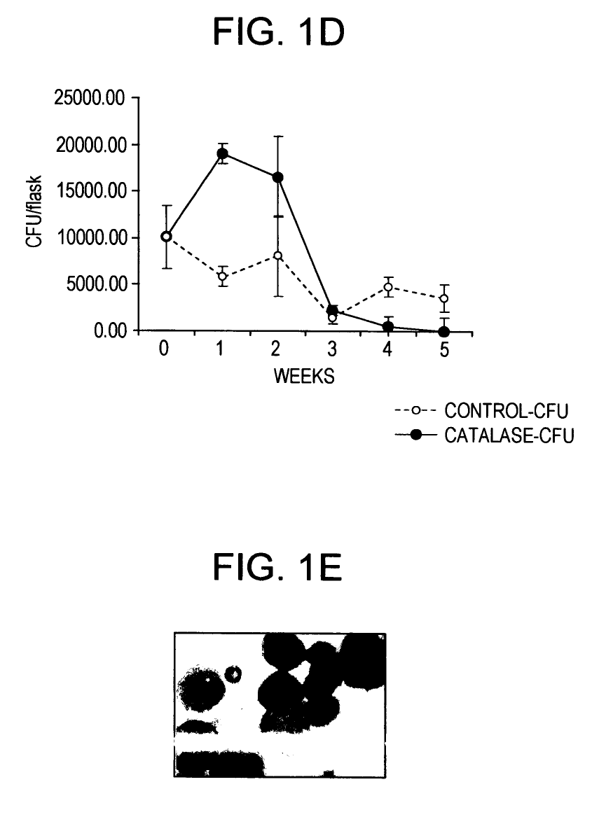

The addition of bovine catalase to murine LTBMC produces dramatic alterations in hematopoiesis. Initially, there is a large (5-10 fold) increase in the number of non-adherent hematopoietic cells in the culture (FIG. 1a). Greater than 90% of these cells are myelocytes, metamyelocytes and granulocytes (FIG. 1b). These are the small refractile cells seen in the photomicrograph (FIG. 1c). This increase is relatively short lived and by the end of 3-4 weeks catalase treated cultures contain only ˜ 1/15^{th} as many hematopoietic cells as do controls (Table 1 and FIG. 1a). Although mature myeloid cells decline after the first week, catalase-containing cultures contain increased numbers of myeloid progenitors (CFU-c) for several weeks (FIG. 1d). At the maximum, the catalase cultures contain up to 10 times the number of granulocytes and ˜5 times the number of clonal progenitors. Because the increase in cell number is short-lived and ends at a time that progenitor number is still high, the net effect is to produce a significant enrichment of CFU among the cells removed from the cultures.

These effects are caused by catalase and not some impurity in the preparation, since neither denatured catalase (heated to 90° C.) nor superoxide dismutase, another ROS scavenger, has any effect on these cultures (data not shown). The addition of the structurally unrelated, but functionally similar catalase from Aspergillus also produces a 2 fold increase in clonal progenitors in LTBMC (Table 1). The effects of bovine catalase are dose-dependent up to 100 μg/ml (Table 1). Because bovine catalase is not stable in these cultures, optimal results were obtained by adding catalase every other day. With time, hematopoiesis diminishes in the catalase-treated cultures. At 3-4 weeks the only hematopoietic cells visible using phase microscopy are large monocytic cells (FIG. 1c left panel). In contrast, the control cultures are actively hematopoietic with many granulocytes (small phase bright cells) and numerous “cobblestone” areas (phase dense cells growing under the stromal layer, marked with *).

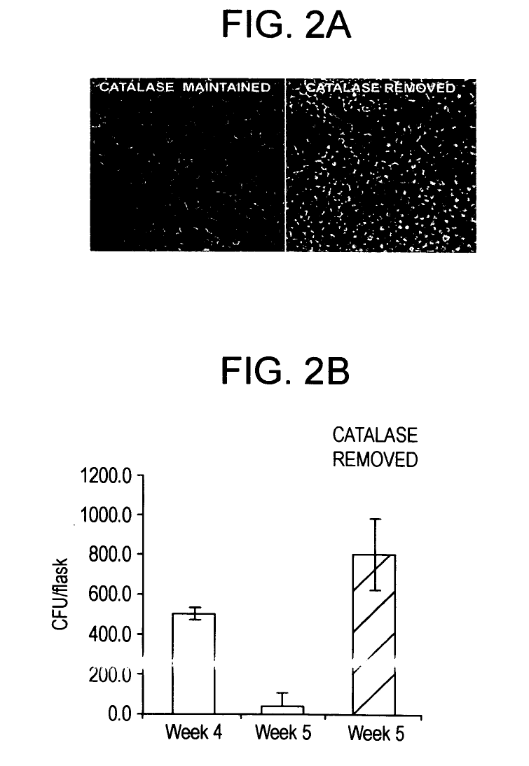

After 4-5 weeks the catalase treated cultures become quiescent; mitosis stops and only monocytes remain as identifiable hematopoietic cells (FIG. 1e and FIG. 2a; left panel). Despite this appearance hematopoietic progenitors are still present. When catalase is removed from the medium, hematopoiesis returns promptly. Within 72 hours “cobblestone areas” are readily observable (FIG. 2a; right panel) and clonal progenitors reappear (FIG. 2b). Within 1 week flow cytometric analyses (see below) of the cultures are indistinguishable from those obtained from cultures of the same age that had not been exposed to catalase (data not shown). In most of the experiments, the stromal cells were irradiated with 450cGy which eliminates any residual hematopoietic cells in the stroma but all of these effects also occurred in cultures in which the stromal cells received (2Gy) or were treated with Mitomycin C (data not shown). Since these treatments inhibit new mRNA by the cells of the preformed stroma, catalase-dependent effects must be due to changes in the progeny of the low-density bone marrow cells.

It has been known for some time that in long-term cultures of mouse bone marrow, more hematopoietic progenitors are found associated with the stromal layer than are found free in the medium. If catalase treatment were only to alter the distribution of the progenitors, more CFU-c would be found in the non-adherent fraction without any overall increase in progenitors. The adherent layer would be depleted of progenitors. To test this possibility, we harvested the progenitor population associated with the adherent layer by trypsinizing the layer (after the removal of the non-adherent cells). A single suspension was prepared, fibroblasts removed by allowing them to reattach to plates as described in the Methods section and the newly released and now non-adherent cells assayed. The results are shown in Table 2. Progenitors in both the non adherent and the adherent compartments were increased by catalase treatment. The changes induced by catalase were in similar magnitude in both locations and the increased number of progenitors found in the non-adherent fraction cannot be the result of redistribution of progenitors from the adherent layer.

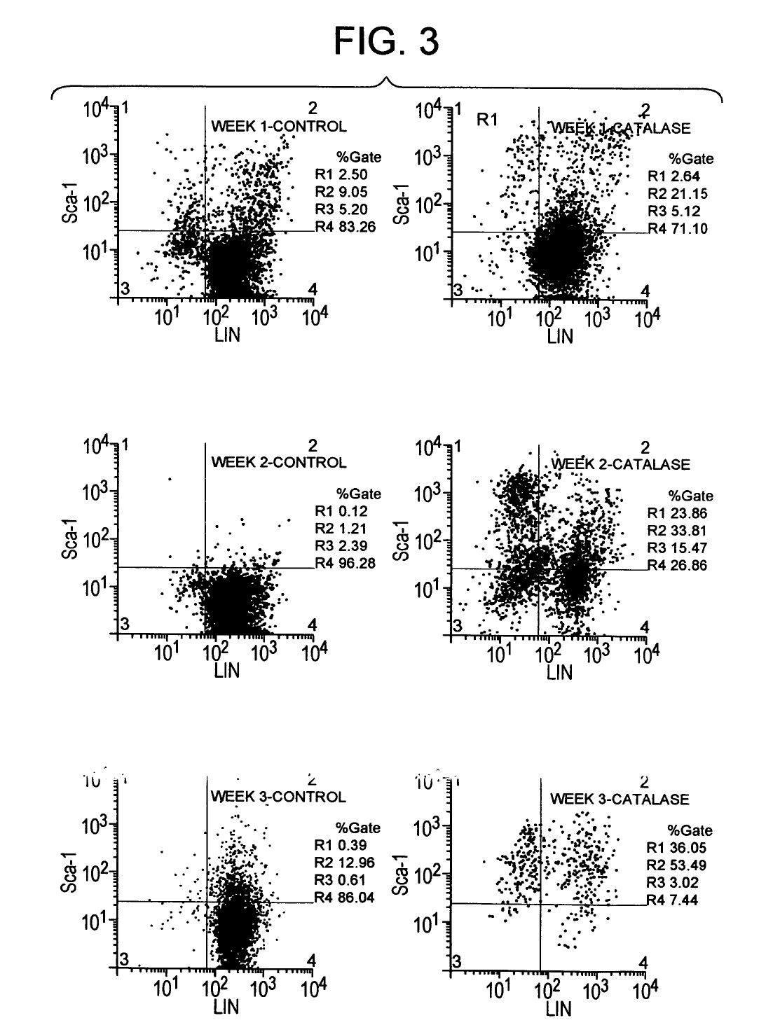

Sca-1+, Gr-1−, Mac-1−, Ter 119−, CD3− and CD4− (Lineage negative: LIN−) progenitor cells accumulate in catalase treated LTBMC (FIG. 3). By the third week of culture >90% of the non-adherent cells were Sca-1+ and a significant proportion of these (36% out of 90%) were LIN−. In terms of absolute numbers, the Sca-1+/LIN− population increases 80 fold from ˜3500 cells (0.5% of 7×10⁵ low-density BMC used to initiate the cultures) to ˜240,000 (24% of 1.05×10⁶) cells after 2 weeks in culture and reaches a maximum of ˜280,000 (36% of 780,000) cells at week 3. If the losses caused by the removal of half of the non adherent cells at each weekly feeding are taken into account the absolute increase in Sca-1+/LIN− negative cells is >500 fold.

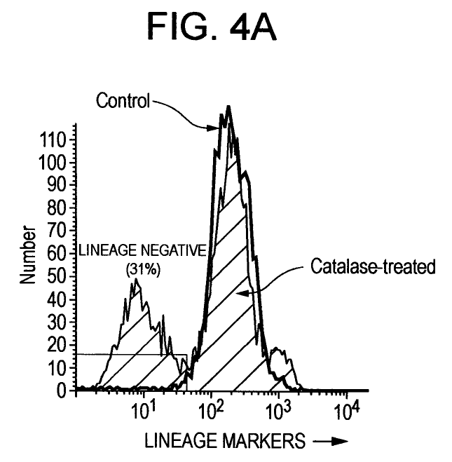

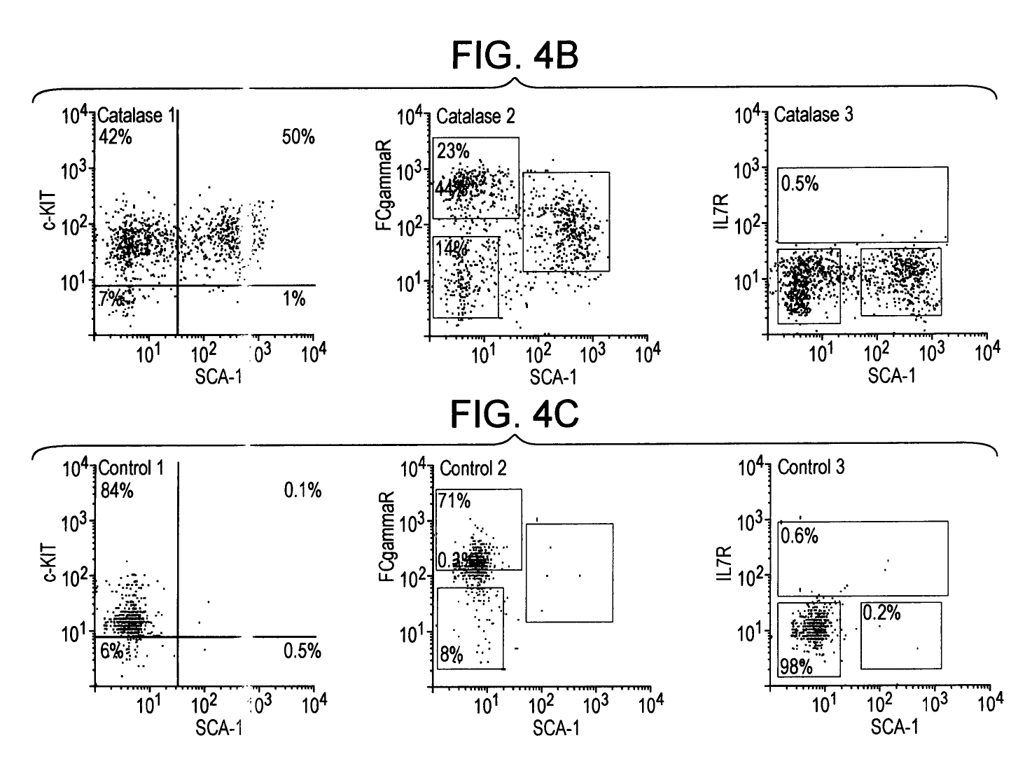

The Sca-1+/LIN− phenotype is characteristic of hematopoietic stem cells and early myeloid progenitors (Orlic, D., Anderson, S., Biesecker, L. G., Sorrentino, B. P., and Bodine, D. M. (1995). Proc. Natl. Acad. Sci. U.S.A. 92:4601-4605; Akashi, K., Traver, D., Miyamoto, T., and Weissman, I. L. (2000). Nature 404:193-197; Kondo, M., Weissman, I. L., and Akashi, K. (1997), Cell. 91:661. It is a heterogeneous population and can be subdivided on the basis of the expression of c-Kit, FcγR (CD16/CD32) and IL7Rα. In another experiment, after 18 days in culture 30.5% of the cells removed from catalase treated cultures were LIN− (FIG. 4a & Table 3). After catalase treatment, 50% of the LIN− cells were Sca-1+ and virtually all of these Sca-1+/LIN− cells are c-Kit positive (49.6% Sca-1+ c-Kit+0 out of 50.5% Sca-1+ cells). Approximately 15% of the cells recovered from LTBMC grown in the presence of catalase have the phenotype described for HSC. This Sca-1+/LIN−/c-Kit+ population also expresses intermediate amounts of FcγR (FIG. 4b center panel) but does not express IL7Rα (FIG. 4b right panel). In addition to cells with a stem cell phenotype these cultures contain many cells (˜23%) with the phenotype ascribed to a “clonogenic common myeloid progenitor” (Akashi, K., Traver, D., Miyamoto, T., and Weissman, I. L. (2000). Nature 404:193-197). These cells, which are Sca-1−/LIN− and FcγR bright (FIG. 4b center panel), are also predominantly c-Kit positive (FIG. 4b left panel). Cells with the phenotype of the “common lymphoid progenitor” (IL7RαSca-1 low,/c-Kit low) (Kondo, M., Weissman, I. L., and Akashi, K. (1997), Cell. 91:661) were not detected (FIGS. 4b and c, right panels). Cells with a stem cell phenotype (Sca-1+/LIN−/c-Kit+) were ˜200-500 times more plentiful in the catalase treated cultures than in the control (FIGS. 4b and c & Table 3).

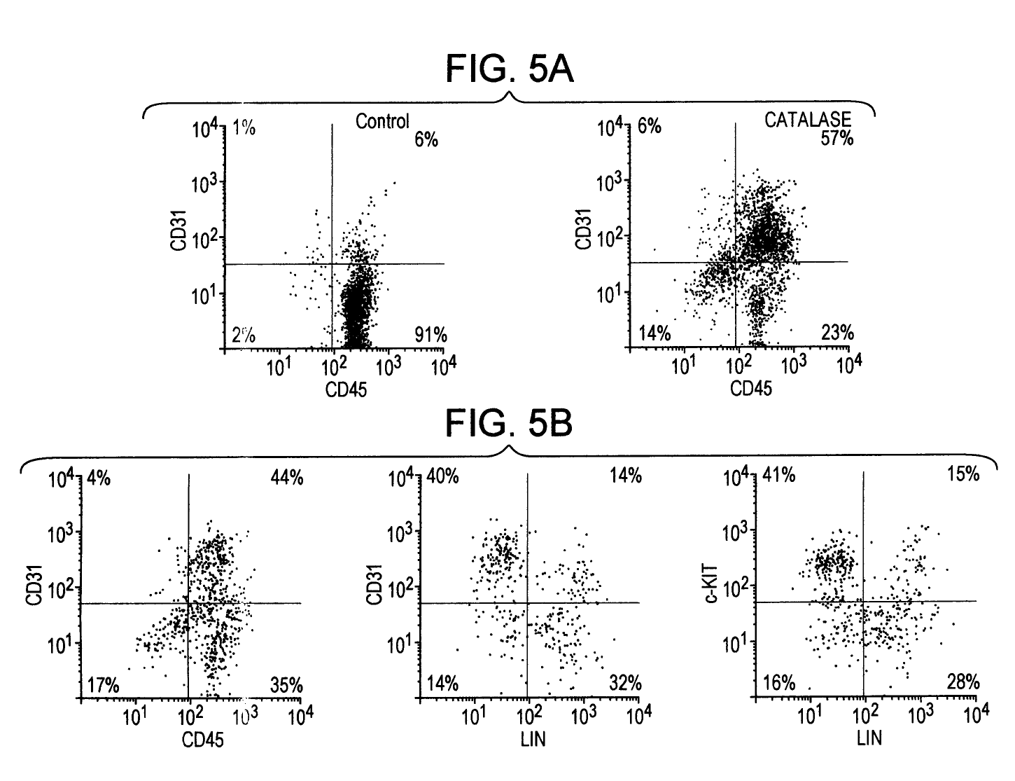

In addition to the increase in Sca-1+ cells, the catalase treated cultures show a significant increase in cells expressing CD31. This is shown in FIG. 5a. 63% of the cells are CD31+ compared to only 7% in the control and 90% (57/63) express Sca-1. Since after 3 weeks in culture, 90% of the non adherent cells express Sca-1 and almost half of these express no lineage markers (Gr-1, Mac-1, Ter 119, CD3 or CD4), we have focused on this Sca-1+/LIN−population.

In another experiment, using 3-color immunofluorescence, the phenotype of these Sca-1+ cells was explored and these results are shown in FIG. 5b. Only results from catalase-treated cultures are shown and the results have been electronically gated on Sca-1+ cells. 79% of the Sca-1+ cells in the culture are CD45+ (Panel A). 44% express both CD45 and CD31 suggesting that they are at a very early stage of development. The results in Panel B show that most of the Sca-1+ CD31+ cells (which are also CD45+ as shown in Panel A) are lineage negative, which is consistent with their immature status. Panel C demonstrates that most of the Sca-1+, LIN-cells are c-KIT+. The Sca-1+, CD45+ phenotype is consistent with an even more immature cell, the hemangioblast. This cell is thought to be able to give rise to both the hematopoietic and vascular endothelial lineages (Goodrich, J. A. et al. (1996), Cell, 84:825-830; Burke, L. J., et al. (2000), FASEB J. 14: 1876-1888; Pelosi, E. et al. (2002), 100: 3203-3208).

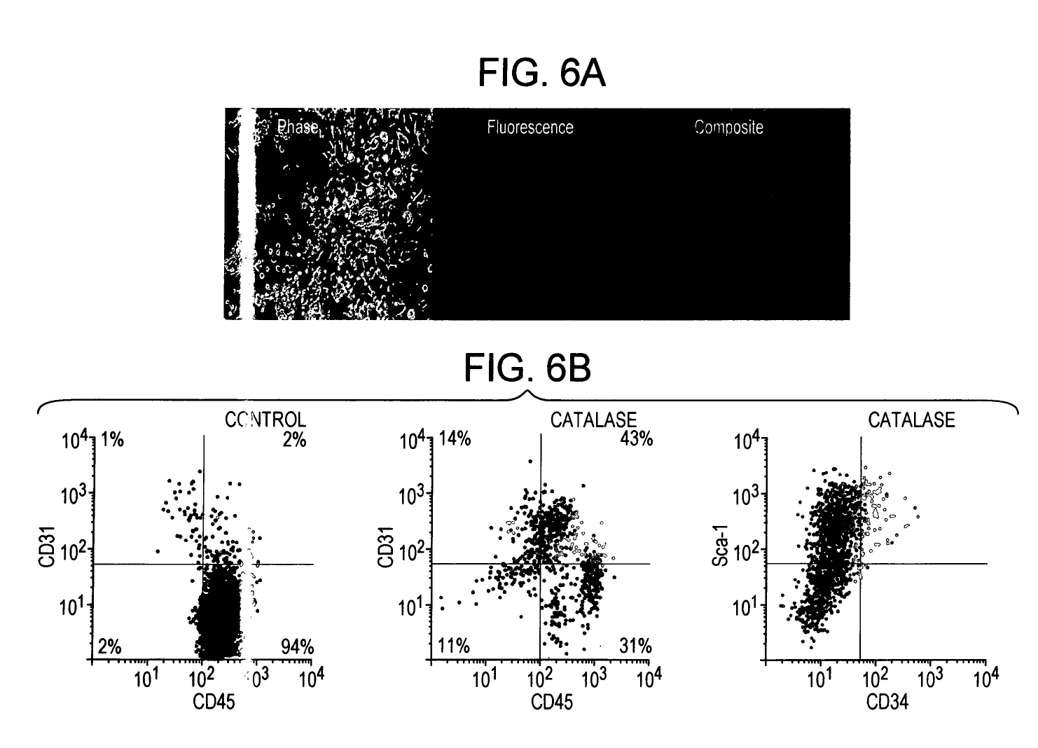

It was also determined that many of the catalase cultured cells express GFP under the control of the Tie-2 promoter. Tie-2 signaling pathways are essential for normal vascular development in the embryo and are important in stabilization of mature blood vessels in the adult (Jones, N. et al. (2001), EMBO Rep. 2: 438-445; Sato, T. N. et al., (1995), Nature 376: 70-74: Hawighorst, T. et al. (2002), Am J Pathol. 160: 1381-1392). When fused to the murine Tie-2 promoter, GFP expression is activated in all endothelial lineage cells. Tie-2 driven GFP expression is also seen in a small number of very immature hematopoietic precursors (Motoike, T. et al. (2000), Genesis 28: 75-81). In these mice, GFP expression in a tissue should correlate with the presence of endothelial cells or their precursors. We made us of this by using BMC from transgenic mice that express the Green Fluorescent Protein (GFP) under the control of the Tie-2 promotor to initiate LT-BMC. FIG. 6a. is a phase-fluorescence composite of a 3-week old culture and shows that both adherent cells and phase dense cells (presumably non adherent) cells express GFP. Note that the “cobblestone areas” which are composed primarily of developing myeloid cells do not express GFP and that Tie-2/GFP expression is seen in both flattened adherent cells and in round cells that do not morphologically resemble endothelial cells.

FIG. 6b. shows a flow cytometric analysis of non-adherent cells produced in these cultures. Catalase treatment results in the accumulation of CD31+, CD45+ cells. 43% of the cells in the catalase-treated culture expressed both CD45 and CD31 (panel B) compared to only 2% in the control cultures (Panel A). Many of these double positive cells also expressed Tie-2 driven GFP (green dots). 87% of the total of Tie-2/GFP positive cells fell in a quadrant used to define CD45+/CD31+ cells. The Tie-2/GFP+ cells also expressed CD34 (Panel C).

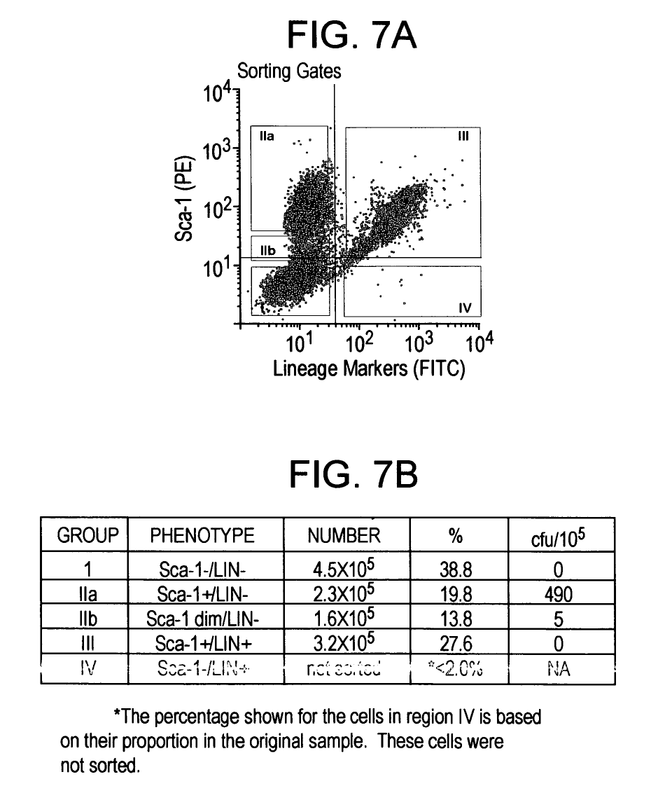

LTBMC grown in the presence of catalase not only contain cells with the phenotype of early members of the hematopoietic lineage, but also are enriched for functional precursors. Cells recovered from the catalase containing cultures were stained with Sca-1 and a cocktail of lineage markers and then sorted into four fractions as shown in FIG. 7. The Sca-1+/LIN− population (Fraction IIA) contains almost all of the small number of CFU-c present in 3 week cultures (FIG. 7b.). The data shown are based on the number of cells recovered after sorting.

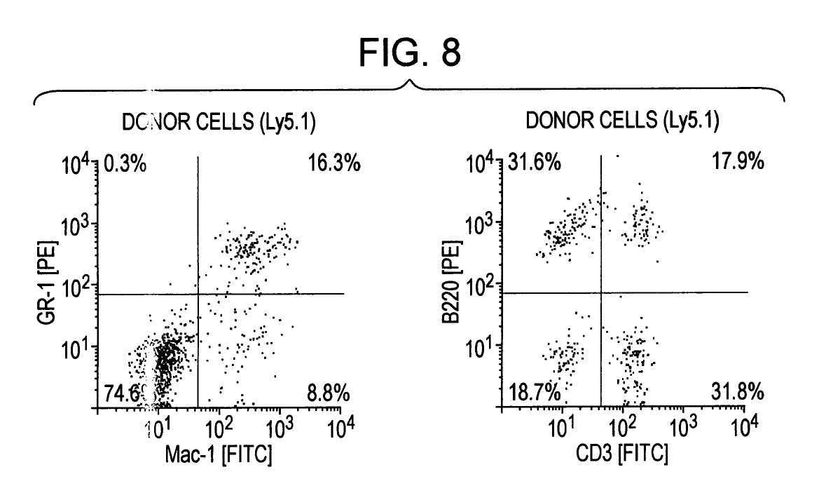

Catalase cultured and control cells were also tested for their ability to restore hematopoiesis in lethally irradiated mice. In these experiments various numbers of cultured Ly5.1 low-density BMC (+catalase for 20 days) were injected into the irradiated Ly5.2 mice along with 25,000 freshly isolated Ly5.2 BMC (5-8 animals/group). The peripheral blood of the recipients was sampled thereafter. The results shown in Table 4 and FIG. 8 demonstrate that the cells cultured in the presence of catalase are better able to provide hematopoietic support than cells in the controls and that the catalase cultured cells are capable of multi-lineage repopulation. Ly5.1 granulocytes (Gr-1+ cells, monocytes (Mac-1+ cells), T-cells (CD3+) and B-cells (B-220+) were all present in the transplanted Ly5.2 mice (FIG. 8).

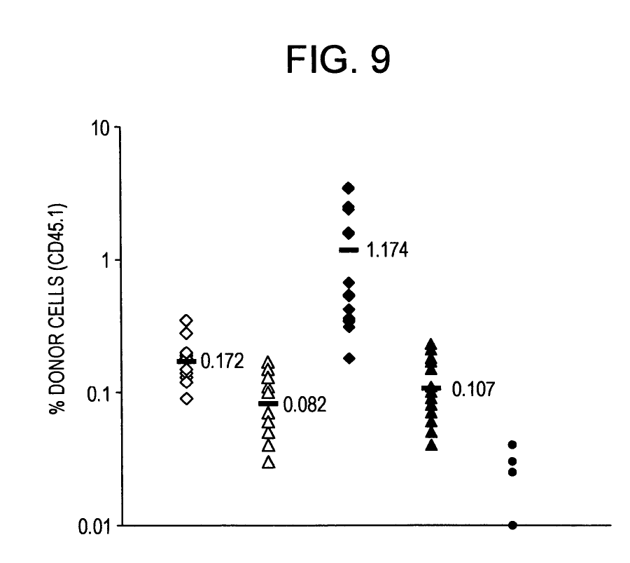

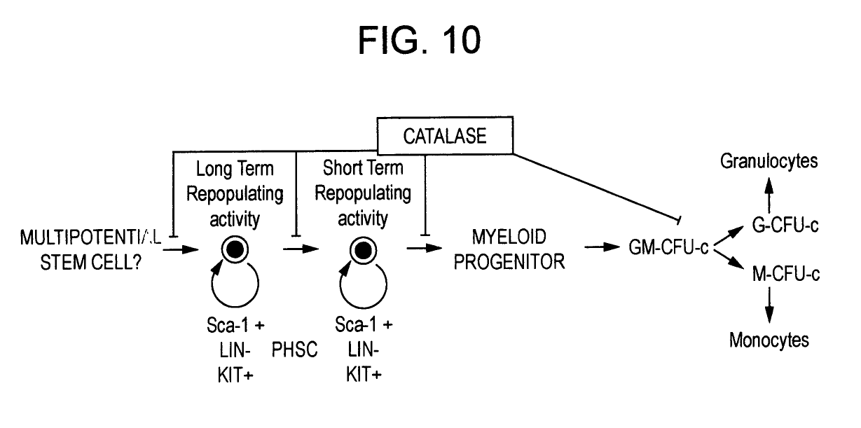

Table 4 shows the repopulation obtained in a series of experiments using cells that had been cultured with or without catalase for 3 weeks to reconstitute lethally irradiated mice. Groups of 8 mice were irradiated and reconstituted with 5,000, 20,000, or 50,000 cells. The extent of repopulation was dose-dependent and at all time points and at cell doses, the cells obtained from catalase-treated cultures had greater repopulating activity than the controls. When 50,000 catalase-treated cells were transferred, a statistically significant increase in repopulation of 3.1, 2.5 and 1.9 fold was noted at 3-4 weeks, 7-8 weeks and 30 weeks, respectively. When 20,000 catalase-treated cells were transferred, similar significant increases in repopulation were noted at 7-8 weeks (4.6 fold), 10-12 weeks (2.3 fold) and 30 weeks (1.8 fold). Since these differences were still apparent when the animals were sacrificed 30 weeks after transplantation, we chose to sacrifice these mice and determine if the cultured cells were capable of reconstituting irradiated mice after a second transfer. As is clear in FIG. 9, cell from the animals reconstituted with catalase-cultured cells contained up to 6.7 times more culture derived cells than did animals reconstituted with control cells.

In addition, a group of mice was transplanted with catalase-cultured cells that had been sorted on the basis of Sca-1 and lineage marker expression; 20,000 of the sorted Sca-1+/LIN− cells obtained from catalase-treated cultures produced extensive repopulation (15.3 to 23.3% over the 3-12 week period), which exceeded the repopulation found in mice that had received an equivalent number of unsorted cells (data not shown) and was greater than that found in mice that had received 50,000 unsorted celsl. Sca-1+/Lin− cells are so infrequent in the control culture that it was impossible to isolate sufficient cells to include in this control. The results indicate the repopulating cells obtained from catalase treated cultures are better able to reconstitute the hematopoietic system of irradiated mice. In the animals reconstituted with the sorted cells, the differences between the control and catalase treated culture tend to increase with time, suggesting that the differences between the two groups are due to better preservation of long term repopulating cells. The results of the sorting experiment show that repopulating cells present in the catalase treated cultures have a phenotype that is similar to that of repopulating cells in uncultured BMC.

Discussion

Catalase profoundly alters hematopoiesis in long-term BMC. Among the early effects of catalase treatment is a dramatic increase in the number of cells expressing Sca-1. The increase is detectable within 48 hours and by the end of 3 weeks the vast majority (65-90%) of the hematopoietic cells express Sca-1. During the second and third weeks of culture, the catalase treated cultures accumulate a large number of cells that do not express antigens characteristic of the mature myeloid or lymphoid lineages. These Sca-1+/LIN− cells have the phenotype of Primitive Hematopoietic Stem Cells (PHSC) (Orlic, D. and Bodine, D. M. (1994), Blood 84:3991-3994). Although all mouse PHSC appear to have the Sca-1+/LIN− phenotype, all cells with this phenotype are not PHSC (Krause, D. S., Theise, N. D., Collector, M. I., Henegariu, O., Hwang, S., Gardner, R., Neutzel, S., and Sharkis, S. J. (2001). Cell 105:369-377; Li, C. L. and Johnson, G. R. (1992). J. Exp. Med. 175:1443-1447). Some cells with this phenotype only provide short-term hematopoietic repopulation and some have no demonstrable stem cell activity (Randall, T. D. and Weissman, I. L. (1998). Blood Cells Molecules and Diseases 16:38-48). Some of the cells that we have grown are able to function as stem cells. Cells capable of both short-term (3-4 weeks) and long-term (30 weeks) repopulation are present and appear to have undergone a significant increase in number. The Sca-1+/LIN− cells that we sorted from catalase-treated LTBMC are both phenotypically and functionally diverse. Only a small proportion (490/105) of them produce colonies in methyl cellulose (FIG. 7b, table). Among the remainder are stem cells capable of long-term multilineage reconstitution and secondary transfer as well as short-term bone marrow repopulation. Within the total Sca-1+/LIN− population we also identified a population that co-expresses c-Kit. In mice, this is the phenotype used to identify PHSC (Akashi, K., Traver, D., Miyamoto, T., and Weissman, I. L. (2000). Nature 404:193-197; Kondo, M., Weissman, I. L., and Akashi, K. (1997). Cell. 91:661.). Many of these Sca-1+/LIN− cells appear to be in the endothelial lineage. The photomicrographs shown in FIG. 6 indicate that in the presence of catalase, the low density BMC used to initiate hematopoiesis in these culture can give rise to adherent cells of the stromal layer. These cells are CD45− and CD31+. Tie-2 is also expressed by CD45+ cells and these cells also express CD31 and Sca-1+. Tie-2 expression is highly correlated with CD34 expression. In the mouse CD34 is primarily an endothelial cell marker and is expressed on only a small subset of hematopoietic cells (Sato, T. et al. (1999), Blood, 94: 2548; Young, P. E. et al. (1995), 85: 96-105). A similar correlation was found with KDR [VEGF2r] expression (data not shown). These results suggest the possibility that these non-adherent cells may be the equivalent of circulating endothelial lineage cells in various states of maturation and that at least some of them may be hemangioblasts. It is also possible that the development of an adherent layer of CD31+ cells may contribute to the formation of a microenvironment that supports hematopoiesis.

After 4-5 weeks the catalase-treated cultures stop releasing differentiated hematopoietic cells into the culture medium and both progenitors and cells with a stem cell phenotype decline. After 4-5 weeks catalase treated cultures resemble aging conventional long term bone marrow cultures. Surprisingly, if the catalase is removed at this time, normal hematopoiesis is restored. The recovery is rapid and within 7-10 days the cultures are indistinguishable from those that had never been treated with catalase. This rapid recovery indicates that the absence of stem cell activity is not the result of toxicity; catalase treatment does not eliminate hematopoietic stem cells but rather, renders them inactive or quiescent. LTC-IC have been shown to enter this state of quiescence spontaneously (Rogers, J. A. et al. (1993), Proc. Natl. Acad. Sci. USA, 90: 5777-5780) and our results suggest that peroxides play a role in its development.

It is likely that the effect of catalase is primarily on the added low-density bone marrow cells in the culture and not upon the preformed stromal layer. Stromal cultures that had been irradiated or mitomycin C treated and thus rendered incapable of new message production, were still able to show the effects of catalase. Furthermore, since hematopoietic progenitors are increased in both the adherent and non-adherent compartments, redistribution of progenitors is not the explanation for the catalase effect.

The processes that determine whether a stem cell undergoes lineage commitment or self-renewal or the ones that govern the progress from progenitor to mature effector cell are complex. The results show that catalase alters this pathway and suggest that several steps in the pathway are subject to ROS regulation. This is shown diagrammatically in FIG. 10.

Thus, neutrophil production in the culture begins immediately (FIG. 1a) but declines to below control levels at a time that progenitors (CFU-c) are increasing (FIG. 1d). In a similar fashion CFU-c decline to below control levels (FIG. 1d) at a time when long-term repopulating cells are more abundant in the catalase treated cultures than in the controls (Table 4). It is possible that catalase may either induce the production of new cytokines or prevent the degradation of those already present, but specific targets are difficult to identify. Medium from catalase treated cultures does not contain increased levels of colony stimulating factors (data not shown) and none of the known growth factors, when added alone, are mitogenic for HSC (Brandt, J. et al. (1992), Blood, 79: 634-641; Shah, A. J. et al. (1996), Blood, 87:3563-3570; Petzer, A. L. et al. (1996), J. Exp. Med. 183:2551-2558; Jacobsen. S. E. W. et al. (1996), Blood, 87:5016-5026; Berardi, A. C. (1995), Science, 267: 104-108). The so called early acting growth factors, 1L-3 and Stem Cell Factor (SCF), primarily stimulate lineage committed progenitors (Berardi, A. C., Wang, A., Levine, J. D., Lopez, P., and Scadden, D. T. (1995). Science 267:104-108.) and although combinations of 4-6 growth factors either in static cultures or in bioreactors permit limited expansion of LTBMC-IC (Brandt, J., Briddell, R. A., Srour, E. F., Leemhuis, T. B., and Hoffman, R. (1992). Blood 79:634-641; Shah, A. J., Smogorzewska, E. M., Hannum, C., and Crooks, G. M. (1996). Blood 87:3563-3570; Petzer, A. L., Zandstra, P. W., Piret, J. M., and Eaves, C. J. (1996). J. Exp. Med. 183:2551-2558; Jacobsen, S. E. W., Veiby, O. P., Myklebust, J., Okkenhaug, C., and Lyman, S. D. (1996). Blood 87:5016-5026), they have not been useful in allowing the production of large numbers of stem cells. It has been suggested that commitment is a random event and that each cell is subject to a finite risk of undergoing commitment as it transits the mitotic cycle (Leary, A. G., Ogawa, M., Strauss, L. C., and Civin, C. I. (1984). J. Clin. Invest. 74:2193-2197; Ikebuchi, K., Ihle, J. N., Hirai, Y., Wong, G. G., Clark, S. C., and Ogawa, M. (1988). Blood 72:2007-2014; Ku, H., Yonemura, Y., Kaushansky, K., and Ogawa, M. (1996). Blood 87:4544-4551). In this view, the role of the known lineage-specific cytokines is to abet the survival of stem cells (Socolovsky, M., Lodish, H. F., and Daley, G. Q. (1998). Proceedings of the National Academy of Sciences 95:6573.) rather than to induce their differentiation. Fairburn et al (Fairbarn, L. J., Cowling, G. J., Reipert, B. M., and Dexter, T. M. (1993). Cell 74:823-832) showed that suppression of apoptosis allows differentiation and development of a multipotent hematopoietic cell line in the absence of added growth factors. The effect of catalase may be to promote stem cell and progenitor survival by inhibiting both differentiation and apoptosis.

The hematopoietic effects may be caused by alterations in the supportive stroma. Our data show that catalase treatment leads to the development of endothelial-like cells in the adherent layer. These cells may be able to provide the equivalent of the “stromal niche” that is believed to be required for HSC survival (Moore, K. A. (2004), Curr. Opin. Hematol. 11: 107-111).

These effects may be caused by catalase and not some impurity in the preparation since 1) heat-inactivated catalase (90° C.×20 minutes) was inactive and the catalase isolated from the mold Aspergillus (tested at concentrations from 2 to 50 ug/ml), was similarly effective. Another ROS scavenger enzyme, superoxide dismutase was without effect in these LTBMC (data not shown).

A direct effect of catalase on the intracellular redox potential may be responsible for the observed effects. It may be possible that the exogenous catalase enters the cells and alters the internal redox potential, leading to alterations in the activity of redox sensitive protein phosphatases and transcription factors. Proteins in the medium enter cells by a variety of pathways but none of these are efficient in transferring intact proteins into the cytosol in the absence of receptor binding (Vives, E., Richard, J. P., Rispal, C., and Lebleu, B. (2003). Curr. Protein Pept. Sci. 4:125-132; van Deurs, B., Roepstorff, K., Hommelgaard, A. M., and Sandvig, K. (2003). Trends Cell Biol. 13:92-100; Qian, Z. M., Li, H., Sun, H., and Ho, K. (2002). Pharmacol. Rev. 54:561-587). This barrier may explain the relatively high concentration of catalase that is required to alter hematopoiesis in culture. Wantanabe (Watanabe, N., Iwamoto, T., Bowen, K. D., Dickinson, D. A., Torres, M., and Forman, H. J. (2003), Biochem. Biophys. Res Commun. 303:287-293) demonstrated that catalase, complexed with the HIV viral product TAT, could enter cells and inhibit serum-induced Elk phosphorylation and anisomycin- and/or MG-132-induced ERK phosphorylation. They showed that free catalase entered cells less well than the complex, but both appeared to bind to cell membranes.

Alternatively, catalase may be acting on peroxides released into the medium by the cells growing in the cultures. Peroxides are freely diffusible and at the appropriate concentration they induce apoptosis rather than necrosis. Pro-apoptotic reactive oxygen intermediates derived from mature phagocytic cells have been shown to play a role in limiting progenitor cell self-renewal (Meagher, R. C., Salvado, A. J., and Wright, D. G. (1988). Blood 72:273-281). Many growth factors appear to promote cell growth by providing an anti-apoptotic signal (Shankar, S. L., O'Guin, K., Cammer, M., McMorris, F. A., Stitt, T. N., Basch, R. S., Varnum, B., and Shafit-Zagardo, B. (2003). J. Neurosci. 23:4208-4218; Walker, A., Ward, C., Dransfield, I., Haslett, C., and Rossi, A. G. (2003). Curr. Drug Targets. Inflamm. Allergy 2:339-347; Streeter, P. R., Dudley, L. Z., and Fleming, W. H. (2003). Exp. Hematol. 31:1119-1125; Broxmeyer, H. E., Kohli, L., Kim, C. H., Lee, Y., Mantel, C., Cooper, S., Hangoc, G., Shaheen, M., Li, X., and Clapp, D. W. (2003). J Leukoc. Biol. 73:630-638; Iwasaki, Y., Ikeda, K., Ichikawa, Y., Igarashi, O., Iwamoto, K., and Kinoshita, M. (2002). Neurol. Res 24:643-646) and it seems possible that catalase, by removing an apoptotic signal, mimics these growth factors. Agents that reduce ROS levels have been shown to be anti apoptotic (Hildeman, D. A., Mitchell, T., Aronow, B., Wojciechowski, S., Kappler, J., and Marrack, P. (2003). Proceedings of the national academy of sciences 100:15035-15040; Lee, J. M. (1998). Oncogene 17:1653-1662).

The fact that catalase profoundly alters hematopoiesis adds to the growing body of evidence that redox signals play an important role in many differentiating systems. H₂O₂ and other ROS are known to function in signal transduction pathways inducing cell death, cell survival, gene activation, and cell movement (Mahadev, K., Zilbering, A., Zhu, L., and Goldstein, B. J. (2001). Journal of Biological Chemistry 276:21938-21942; Sen, C. K. and Packer, L. (1996). FASEB J 10:709-720; Morel, Y. and Barouki, R. (1999). Biochem. J 342 Pt 3:481-96.:481-496) and activation with GM-CSF and other growth factors including IL-3, SF, and TPO is associated with alterations in levels of H₂O₂(Sattler, M., Winkler, T., Verma, S., Byrne, C. H., Shrikhande, G., Salgia, R., and Griffin, J. D. (1999). Blood 93:2928-2935). Selectively increasing intracellular ROS by adding H₂O₂ induces tyrosine phosphorylation and other signaling events. Conversely treatment of cells with reducing agents suppressed both H₂O₂ formation and GM-CSF-activated signal transduction, indicating that peroxides may play a role in growth factor signal transduction. Reducing agents such as pyrrolidine dithiocarbamate (PDTC), 2-mercaptoethanol, and N-acetyl cysteine suppress growth of many hematopoietic cell lines, (Sattler, M., Winkler, T., Verma, S., Byrne, C. H., Shrikhande, G., Salgia, R., and Griffin, J. D. (1999). Blood 93:2928-2935). The interaction of hematopoietic growth factors (HGF) with ROS is complex since not only does treatment with HGF increase intracellular ROS levels, but exogenous H₂O₂ produced concentration-dependent induction of GM-CSF mRNA in some cells (Tibbles, L. A. and Woodgett, J. R. (1999). Cell Mol. Life Sci. 55:1230-1254).

Catalase treatment replicates the effects of the so-called “stem cell niche” of the bone marrow, promoting myeloid differentiation without lymphopoiesis and inducing a quiescent state in PHSC (Calvi, L. M., Adams, G. B., Weibrecht, K. W., Weber, J. M., Olson, D. P., Knight, M. C., Martin, R. P., Schipani, E., Divieti, P., Bringhurst, F. R. et al. (2003). Nature 425:841-846; Zhang, J., Niu, C., Ye, L., Huang, H., He, X., Tong, W. G., Ross, J., Haug, J., Johnson, T., Feng, J. Q. et al. (2003). Nature 425:836-841). In addition to highlighting the importance of peroxide levels in regulating hematopoietic differentiation, these cultures may provide a unique source of undifferentiated hematopoietic progenitors. These Sca-1+/Lin−progenitors have both serological and functional properties that indicate that this population is highly enriched for hematopoietic stem cells and that these cells have increased in number by up to five hundred fold. While considerable success has been achieved in growing stem cells from mice that have been genetically modified (Krosl, J., Beslu, N., Mayotte, N., Humphries, R. K., and Sauvageau, G. (2003). Immunity. 18:561-571; Bhardwaj, G., Murdoch, B., Wu, D., Baker, D. P., Williams, K. P., Chadwick, K., Ling, L. E., Karanu, F. N., and Bhatia, M. (2001). Nat. Immunol 2:172-180; Willert, K., Brown, J. D., Danenberg, E., Duncan, A. W., Weissman, I. L., Reya, T., Yates, J. R., III, and Nusse, R. (2003). Nature 423:448-452; Antonchuk, J., Sauvageau, G., and Humphries, R. K. (2002). Cell 109:39-45), unmodified cells have proved more difficult to grow. The best results reported previously are the 4-6 fold expansion achieved using human TAT-HOXB4 protein (Krosl, J., Austin, P., Beslu, N., Kroon, E., Humphries, R. K., and Sauvageau, G. (2003). Nat. Med. 9:1428-1432) and a similar expansion of SCID repopulating cells in cord blood cells cultured with a copper chelator (Peled, T. et al. (2004), Exp. Hematol. 32: 547-555). Our results indicate that much greater expansion is possible using the methods described herein.

Summary of Results

The studies presented herein demonstrate that the addition of catalase to the medium of long-term bone marrow cultures (LTBMC) significantly alters hematopoiesis in these cultures. The results are complex and may change with time. The initial effect may be to increase the production of granulocytes. As the cultures develop, maturation of hematopoietic progenitor cells (HPC) may be retarded and Sca-1+/LIN− cells accumulate. After ˜3 weeks in culture these cells, which have the phenotype of primitive hematopoietic stem cells (PHSC), have expanded by several hundred fold. Catalase-treated cultures also provide a remarkable source of early hematopoietic progenitors which may prove useful clinically.

TABLE 1
Dose-Response of the effect of catalase on mouse LTBMC.

| | Conc. | Replen- | Cells/flask | CFU/ | |
|---|---|---|---|---|---|
| CATALASE | (ug/ml) | ished | (×10⁶)* | 1000* | CFU/flask** |
| none | 0 | 3/week | 4.7 | .35 | 1650 +/− 366 |
| Bovine | 50 | 3/week | 1.2 | 5.61 | 6390 +/− 515 |
| Bovine | 100 | 3/week | 1.0 | 12.11 | 12110 +/− 663 |
| Bovine | 200 | 3/week | 1.4 | 6.11 | 8870 +/− 1610 |
| none | 0 | 1/week | 1.3 | 0.29 | 377 +/− 103 |
| Aspergillus | 10 | 1/week | 0.35 | 1.51 | 528 +/− na |
| Aspergillus | 30 | 1/week | 0.40 | 1.95 | 780 +/− 128 |
| Aspergillus | 60 | 1/week | 0.25 | 2.45 | 604 +/− 320 |

*Total number of nonadherent cells in the cultures and colony forming units per culture measured at week 2 (for bovine catalase) and week 3 (aspergillus catalase). The results shown are the mean of duplicate or triplicate determinations.
**CFU per cells plated in methylcellulose × cells recovered per flask +/− S.E.M.

TABLE 2
Effect of Catalase on the Distribution of Progenitors in LTBMC

| | | Cell | CFU/ | CFU/ |
|---|---|---|---|---|
| | | count/culture | 100,000 | culture |
| CONTROL | Non-adherent | 2.6 × 10⁶ | 32 | 832 |
| CONTROL | Non-adherent | 2.4 × 10⁶ | 20 | 480 |
| CATALASE | Non-adherent | 2.3 × 10⁶ | 180 | 4140 |
| CATALASE | Non-adherent | 2.0 × 10⁶ | 370 | 7400 |
| CONTROL | Adherent | 2.5 × 10⁶ | 62 | 1550 |
| CONTROL | Adherent | 4.1 × 10⁶ | 32 | 1312 |
| CATALASE | Adherent | 3.1 × 10⁶ | 250 | 7750 |
| CATALASE | Adherent | 4.0 × 10⁶ | 542 | 21680 |

TABLE 3
Recovery of Sca-1+ and c-Kit+ cells from catalase treated and control cultures.

| | CONTROL | | | CATALASE | | |
|---|---|---|---|---|---|---|
| | Cell Number | % Total | % of LIN- | Cell Number | % Total | % of LIN- |
| TOTAL | 5600000 | 100.0% | — | 260000 | 100.0% | — |
| LINEAGE NEGATIVE | 140000 | 2.5% | 100.0% | 79248 | 30.5% | 100.0% |
| SCA-1+ | 840 | 0.0% | 0.6% | 40020 | 15.5% | 50.5% |
| c-KIT+ | 117320 | 2.1% | 83.8% | 72670 | 28.0% | 91.7% |
| SCA-1+/c-KIT+ | 211 | 0.0% | 0.15% | 39307 | 15.1% | 49.6% |

| | |
|---|---|
| RATIO: SCA-1+/c-KIT+ [CATALASE]/SCA-1+/c-KIT+ [Control] | 186.3 |

TABLE 4
Proportion of CD45.1 (donor derived) Cells in Peripheral Blood.

| | Number of | Percentage CD45.1 Donor Cells | | | |
|---|---|---|---|---|---|
| | Cells | (Mean +/− S.E.M.) | | | |
| Group+ | Injected* | Time After Transplantation | | | |
| | | 4 Weeks | 8 Weeks | 12 Weeks | 30 Weeks |
| LD BMC | 50,000 | 2.8 +/− 0.3 | 6.4 +/− 0.6 | 7.0 +/− 1.1 | 5.4 +/− 0.6 |
| (cultured 3 weeks | 20,000 | 2.8 +/− 0.7 | 4.5 +/− 0.4 | 4.9 +/− 0.7 | 4.8 +/− 0.8 |
| without catalase) | 5,000 | 1.8 +/− 0.8 | 1.2 +/− 0.9 | 2.1 +/− 0.2 | ND |
| LD BMC | 50,000 | 7.1 +/− 1.8^{a} | 11.3 +/− 1.6^{a} | 22.6 +/− 6.6^{a,b} | 10.4 +/− 1.2* |
| (cultured 3 | 20,000 | 5.4 +/− 1.0 | 12.9 +/− 2.2^{a} | 7.1 +/− 1.4 | 8.2 +/− 0.8* |
| weeks with | 5,000 | 2.7 +/− 0.5 | 3.1 +/− 0.9 | 5.3 +/− 0.3 | ND |
| catalase) | | | | | |
| | | 3 Weeks | 7 Weeks | 10 Weeks | |
| Sorted Sca- | 20,000 | 15.3 +/− 4.9 | 23.0 +/− 8.0 | 23.3 +/− 4.6 | ND |
| 1+/LIN- | | | | | |
| (from catalase | | | | | |
| treated culture) | | | | | |

+Each group contained 9 or 10 mice and each sample was analyzed in duplicate.
*Each Mouse also received 25,000 fresh syngeneic (CD45.2) bone marrow cells (BMCs) to assure survival after irradiation.
^{a}Value differs from the catalase-free control culture with a p value of <0.05.
^{b}This value includes the results from a single mouse that had >70% donor cells. If that outlying animal is eliminated the resultant value would be 15.5 +/− 4.1%.
ND Not done.

TABLE 5
GenBank Accession Numbers for Growth Factors

| | | SEQ ID | | SEQ ID |
|---|---|---|---|---|
| | Mouse | NO | Human | NO |
| Granulocyte Macrophage | NM_007780 | 1 | NM_000758 | 2 |
| Colony Stimulating | | | | |
| Factor | | | | |
| (GM-CSF) | | | | |
| Granulocyte | NM_009971 | 3 | NM_172220 | 4 |
| Colony Stimulating | | | | |
| Factor (G-CSF) | | | | |
| Macrophage | NM_007778 | 5 | NM_172212 | 6 |
| Colony Stimulating | | | | |
| Factor (M-CSF) | | | | |
| Interleukin-3 (IL-3) | NM_010556 | 7 | NM_000588 | 8 |
| Interleukin-7 (IL-7) | NM_008371 | 9 | NM_000880 | 10 |
| Erythropoietin (EPO) | NM_007942 | 11 | NM_000799 | 12 |
| Thrombopoietin (TPO) | NM_009379 | 13 | NM_199356 | 14 |
| Interleukin-5 (IL-5) | NM_010558 | 15 | NM_000879 | 16 |

What is claimed is:

1. A method for culturing, growing and/or maintaining stem cells or derivatives thereof comprising the steps of:
a) providing a population comprised of said stem cells or derivatives thereof; and
b) stimulating the growth of said stem cells or derivatives thereof by incubation of said stem cells in medium supplemented with a growth promoting and/or maintenance promoting amount of catalase; wherein said stimulating promotes the growth of and maintains more stem cells or derivatives thereof in said population as compared to a population which has not been stimulated with a growth promoting and/or maintenance promoting amount of catalase.

2. The method of claim 1, wherein said stem cells or derivatives thereof are hematopoietic stem cells (HSC), hematopoietic progenitor cells (HPC), hemangioblasts and endothelial cell precursors (EPC).

3. The method of claim 2, wherein said hematopoietic stem cells or derivatives thereof are capable of multilineage repopulation in vitro or in vivo.

4. The method of claim 3, wherein said repopulation results in the growth and expansion of cells selected from the group consisting of granulocytes, monocytes, T cells and B cells.

5. The method of claim 1, wherein said method results in granulopoiesis during the first week of catalase treatment, followed by a decline by the third week.

6. The method of claim 1, wherein said method further results in a large increase in clonal progenitors (CFU-c).

7. The method of claim 1, wherein said method results in the detectable levels of cells expressing Sca-1 within the first week of culture in the presence of catalase.

8. The method of claim 7, wherein said method further results in about 65 to 90% of the cells expressing Sca-1.

9. The method of claim 8, wherein said method further results in an increase in the number of cells having the Sca-1+/LIN− phenotype after two to three weeks in culture with catalase.

10. The method of claim 9, wherein said method further results in quiescence of hematopoietic stem cells and progenitor cells after about four to five weeks in culture in the presence of catalase.

11. The method of claim 10, wherein said method further results in restoration of hematopoiesis upon removal of catalase.

12. The method of claim 1, wherein said method results in the accumulation of long-term bone marrow initiating cells (LTBMiC).

13. The method of claim 12, wherein the long-term bone marrow initiating cells (LTBMiC) that accumulate are Sca-1+, Gr-1−, Mac-1−, Ter119−, CD3− and CD4− (lineage negative or LIN− cells).

14. The method of claim 12, wherein about 15% of the cells from LTBMC grown in catalase are Sca-1+/LIN−/c-Kit+ and which also express intermediate amounts of FcγR but do not express IL7Rα.

15. The method of claim 12, wherein about 25% of the cells from LTBMC have a phenotype characteristic of a clonogenic common myeloid progenitor cell.

16. The method of claim 15, wherein said clonogenic common myeloid progenitor cell is Sca-1−/LIN−, FcγR bright and predominantly cKit+.

17. The method of claim 12, wherein said method results in the absence of cells having the characteristics of a common lymphoid progenitor in vitro, and wherein said cells upon transfer to an animal result in growth of lymphoid cells in vivo.

18. The method of claim 17, wherein said common lymphoid progenitor cells are IL7Rα+/Sca-1 low and c-Kit low.

19. The method of claim 1, wherein said stem cells or derivatives thereof grown in the presence of catalase are about 200 to 500 times more plentiful than stem cells grown in the absence of catalase.

20. An isolated pure population of stem cells or derivatives thereof, said cells grown by the method of claim 1.

21. The population of claim 20 wherein said stem cells or derivatives thereof are isolated from a mammal selected from the group consisting of human and non-human primates, rodents, equines, canines, felines, bovines, porcines, ovines, and lagomorphs.

22. The stem cells of either one of claims 1 or 21, wherein said stem cells or derivatives thereof are derived from an autologous or heterologous donor or from cord blood.

23. The population of claim 20 wherein said stem cells or derivatives thereof are capable of differentiating into granulocytes, monocytes, T cells and B cells.

24. A method for treating a subject suffering from a disease or disability which is causally related to or following from the lack or insufficiency of cells of a particular lineage, comprising administering to said subject in need of such treatment the stem cells or derivatives thereof grown by the method of claim 1 in an amount effective for treating the disease or disability.

25. A method of treating a disease that results from insufficient growth and/or differentiation of hematopoietic stem cells, hematopoietic progenitors, or a combination thereof, said method comprising administering the stem cells or derivatives thereof prepared by the method of claim 1.

26. The method of claim 25, wherein said disease or disability is selected from the group consisting of anemias, leukemia, lymphoma, inherited blood disorders, inherited metabolic disorders and diseases or treatments resulting in an immunodeficiency.

27. The method of claim 24 wherein said stem cells or derivatives thereof are caused to proliferate and differentiate in vitro prior to being administered.

28. The method of claim 24 wherein said stem cells or derivatives thereof are from a heterologous or an autologous donor.

29. The method of claim 28 wherein said donor is a fetus, a juvenile or an adult.

30. The method of claim 24 wherein said stem cells are obtained from umbilical cord blood.

31. The method of claim 24 wherein said stem cells or derivatives thereof are administered locally to the site of tissue damage.

32. The method of claim 24 wherein said stem cells or derivatives thereof are administered in an encapsulation device.

33. The method of claim 24 wherein said derivatives thereof are obtained by genetic transduction of stem cells.

34. A method for treating a blood disorder that results in anemia or in an immunodeficiency in a mammal, or an inherited metabolic disease, or an inherited immune disorder, or an inherited red cell disorder and marrow failure, comprising the steps of:
a) providing a pure population of stem cells grown by the method of claim 1;
b) genetically transforming said stem cells with a gene encoding a growth factor, or substance that provides for enhanced proliferation and/or differentiation of the stem cells resulting in a transformed population of stem cells that express said growth factor; and
c) administering an effective amount of said transformed population of stem cells to said mammal.

35. The method of claim 34, wherein said inherited metabolic disorders may be selected from, but not limited to, the group consisting of adrenoleukodystrophy, Hurler's Syndrome, Pompe's disease, metachromatic leukodystrophy, and osteopetrosis.

36. The method of claim 34, wherein said inherited immune cell disorders may be selected from, but not limited to, the group consisting of Severe Combined Immunodeficiency, ADA deficiency, and Wiskott-Aldrich Syndrome.

37. The method of claim 34, wherein said inherited red cell disorders may be selected from, but not limited to, the group consisting of pure red cell aplasia, sickle cell disease, beta thalassemia, aplastic anemia and Fanconi anemia.
